(19) United States
(12) Patent Application Publication
(10) Pub. No.: US 2011/0015220 A1
(43) Pub. Date: Jan. 20, 2011

(54) MIXED ORL1/MU-AGONISTS FOR THE TREATMENT OF PAIN

(75) Inventors: KLAUS LINZ, WACHTBERG (DE); BABETTE-YVONNE KÖGEL, LANGERWEHE (DE); WOLFGANG SCHRÖDER, AACHEN (DE); THOMAS CHRISTOPH, AACHEN (DE); JEAN DE VRY, STOLBERG (DE); ELMAR FRIDERICHS, STOLBERG (DE)

Correspondence Address: Briscoe, Kurt G.;Norris McLaughlin & Marcus, PA, 875 Third Avenue, 8th Floor, New York, NY, 10022, US

(73) Assignee: GRUNENTHAL GMBH, AACHEN (DE)

(21) Appl. No.: 12/887,102

(22) Filed: Sep. 21, 2010

Related U.S. Application Data
(62) Division of application No. 11/864,080, filed on Sep. 28, 2007.

(30) Foreign Application Priority Data
Sep. 29, 2006 (DE) 10 2006 046 745.0

(51) Int. Cl. A61K 31/438; A61K 31/407; A61P 29/00; A61P 23/02; A61P 3/10

(52) U.S. Cl. 514/278; 514/409

(57) ABSTRACT

The invention relates to the use of compounds which exhibit an affinity for the μ-opioid receptor of at least 100 nM (Kᵢ value, human) and an affinity for the ORL-1 receptor, wherein the ratio between the affinities ORL1/μdefined as 1/[K_{i(ORL1)}/K_{i(μ)}] is from 0.1 to 30, for the treatment of pain.

In addition to acute pain, which is of limited duration and generally rapidly subsides after removing the triggering stimuli, chronic pain in particular constitutes a challenge to medical science. Acute pain phenomena due to the stimulation of intact nociceptors have a warning function to preserve physical integrity. The subsequent responses to avoid pain provide protection from injury. Chronic pain has lost this protective function. A pain disorder is then present. Chronic pain may here be subdivided into two major groups. Pathophysiological nociceptor pain is caused after tissue trauma by the stimulation of intact nociceptors. Such pain in particular includes chronic inflammatory pain. In contrast, pain arising due to damage to the nerves themselves is known as neuropathic pain.

The changeover from acute pain to chronic pain may occur within hours. Pain treatment during and following surgery is, for example, affected by this. Although doctors are now highly aware of the treatment of acute pain, severe limits apply to the treatment of postoperative pain (Power, Brit. J. Anaesth., 2005, 95, 43-51). Acute pain may become chronic peripherally and in the CNS by means of pathophysiological processes subsequent to tissue damage, for example, surgery. The association between tissue damage, acute postoperative pain and the development of chronic pain has been thoroughly investigated, it being possible to regard the severity of the acute pain as a predictive factor for the duration of the chronic pain (Power, Brit. J. Anaesth., 2005, 95, 43-51). Merely for this reason, satisfactory treatment of acute pain is essential.

One problem in combating acute pain is the side-effects, in particular, respiratory depression, of μ-opioids, such as morphine or fentanyl, which are highly effective against acute pain. Since this side-effect occasionally results in fatalities in patients having just undergone surgery, the medicaments are in many cases not given in sufficient quantity to combat the pain satisfactorily. On the other hand, it is now inconceivable to treat postoperative pain without opioids. However, the fear of respiratory depression and further side-effects typical of μ-opioids in many cases results in opioids being used to an inadequate extent in severe pain, for example, in cancer patients (Davis et al., Respiratory Care Journal 1999, 44 (1)). Furthermore, the risk of respiratory depression occurring after administration of opioids is higher in older people than in younger people. In fact, the risk of developing respiratory depression rises distinctly in people from 60 years of age (Cepeda et al., Clinical Pharmacology and Therapeutics 2003, 74, 102-112). There is thus an urgent need for new medicaments for the treatment of pain in which respiratory depression is reduced.

However, as has already been mentioned, the treatment of chronic pain is a relatively large challenge because, while commercially available medicaments are indeed in some cases highly active against acute pain, in many cases they do not however provide satisfactory pain treatment in chronic pain.

### Inflammatory Pain

In addition to reddening, swelling, overheating and impaired function, pain is one of the five cardinal symptoms of inflammation. Inflammatory processes are among the most important mechanisms involved in the genesis of pain. Typical inflammatory pain is triggered by the release of bradykinin, histamine and prostaglandins with tissue acidification and exudate pressure on the nociceptors. Unlike other kinds of sensory perception, nociception is not subject to habituation. Instead, preceding pain impulses may amplify the processing of subsequent stimuli resulting in sensitization. If an increased influx of pain impulses to the central nervous system occurs, for example due to long-term activation of nociceptors in the inflamed tissue, lasting sensitization phenomena occur in the central synapses. These central sensitization phenomena are manifested in an increase in spontaneous activity and in stronger responses to stimulation of central neurons, the receptive fields of which likewise become larger (Coderre et al., Pain 1993, 52, 259-285). These changes to the response behavior of central neurons may contribute to spontaneous pain and hyperalgesia (increased perception of pain in response to a noxious stimulus), which are typical of inflamed tissue (Yaksh et al., PNAS 1999, 96, 7680-7686).

One of the most important processes in inflammation is the occurrence of arachidonic acid metabolites. These compounds do not activate nociceptors directly, but instead reduce the stimulus propagation threshold of the C fibers and so sensitize them to other stimuli. Nonsteroidal antiinflammatory agents (NSAIDs) have in particular proved effective in treating inflammatory pain, as they block arachidonic acid breakdown (Dickensen, A., International Congress and Symposium Series—Royal Society of Medicine (2000), 246, 47-54). However, their use in long-term treatment of chronic pain is limited by sometimes considerable unwanted effects, such as gastroenteral ulcers or toxic kidney damage.

Inhibitory control of stimulus propagation is, however, also of significance in the treatment of inflammatory pain. μ-Opioids are the most important members of this class. Chronic pancreatitis, for example, is accompanied by pain, which is among the clinically most difficult pain states to treat. Administration of NSAIDs possibly only slightly reduces the pain, but results in an elevated risk due to the increased risk of bleeding. The next step is generally treatment with μ-opioids. Dependency on narcotic analgesics is widespread in patients suffering from this condition (Vercauteren et al., Acta Anaesthesiologica Belgium 1994, 45, 99-105). There is therefore an urgent need for compounds which are highly active against inflammatory pain and have reduced potential for dependency.

### Neuropathic Pain

Neuropathic pain occurs when peripheral nerves suffer mechanical, metabolic or inflammatory damage. The pain pictures which arise as a result are predominantly characterized by the occurrence of spontaneous pain, hyperalgesia and allodynia (pain which is triggered even by non-noxious stimuli). Increased expression of Na+ channels and thus spontaneous activity in the damaged axons and their neighboring axons occurs as a consequence of the lesions (England et al., Neurology 1996, 47, 272-276). Excitability of the neurons is raised and they respond to incoming stimuli with an increased discharge frequency. Increased sensitivity to pain is the result, which contributes to the development of hyperalgesia and spontaneous pain (Baron, Clin. J. Pain 2000; 16 (2 Suppl.), 12-20).

The causes and severities and therefore also the treatment needs of neuropathic pain are diverse. They arise as a consequence of injuries or diseases of the brain, spinal chord or peripheral nerves. Causes can be operations, e.g. phantom pain following amputation, stroke, multiple sclerosis, injuries to the spinal chord, alcohol or medicament abuse or other toxins, cancer diseases and also metabolic diseases, such as diabetes, gout, renal insufficiency or cirrhosis of the liver, or infectious diseases, such as mononucleosis, ehrlichiosis, typhus, diphtheria, HIV, syphilis or borrelioses. The pain experience has very different signs and symptoms which can change in number and intensity over time. Paradoxically, patients suffering from neuropathic pain described a decrease or disturbance in the perception of acute pain with a simultaneous increase in the neuropathic pain. Typical symptoms of neuropathic pain are described as tingling, burning, shooting, electrifying or radiant. Tricyclic antidepressants and anticonvulsives, used as monotherapy or also in combination with opioids, are among the basic pharmacological treatments for neuropathic pain. These medicaments usually alleviate the pain only to a certain extent, with freedom from pain often not being achieved. The side-effects which frequently occur often stand in the way of increasing the dose of the medicaments in order to achieve adequate pain relief. In fact, satisfactory treatment of neuropathic pain frequently entails a higher dosage of a μ-opioid than does the treatment of acute pain, whereby the side-effects become even more significant. This problem is further increased by the onset of the development of tolerance, which is typical of μ-opioids, and the associated need to increase the dose. To summarize, it may be concluded that neuropathic pain is today difficult to treat and is only partially alleviated by high doses of μ-opioids (Saudi Pharm. J. 2002, 10 (3), 73-85). There is thus an urgent requirement for medicaments for treating chronic pain, the dose of which does not have to be increased until intolerable side-effects occur, in order to provide satisfactory treatment of pain.

In recent decades, various other modes of action for the treatment of chronic pain which do not exhibit the side-effects typical of opioids have been proposed and implemented. Accordingly, moderately severe to severe chronic pain is treated with antidepressants which, apart from raising mood, also exhibit an analgesic action. However, no mode of action has hitherto been able to displace μ-opioids from their central significance in the treatment of pain. One of the principal reasons is the hitherto unequalled effectiveness of μ-opioids. However, apart from respiratory depression, μ-opioids also exhibit further disadvantages:

### a) Opioid-Induced Hyperalgesia

It has been known for more than 100 years that increased perception of pain is one of the symptoms of opioid withdrawal. Today, the occurrence of pain symptoms is among the criteria for diagnosing opioid withdrawal (Angst et al., Anesthesiology 2006, 104, 570-587). A growing number of animal and human studies have shown that, under certain circumstances, μ-opioids may cause changes in the perception of pain which lead to hyperalgesia (increased perception of pain after a painful stimulus). These studies have shown that the phenomenon of opioid-induced hyperalgesia occurs after both brief and chronic opioid administration (Pud et al., Drug and Alcohol Dependence 2006, 218-223). It is known, for example, that patients who receive anaesthesia with an elevated opioid content require around three times the amount of opioids postoperatively in comparison with patients who receive hypnotic anaesthesia. This clear effect also restricts the safe use of μ-opioids, since the consequently necessary increase in dose also increases the significance of side-effects such as respiratory depression. However, since the treatment of severe pain is today inconceivable without opioids, there is an urgent need for medicaments which do not themselves give rise to increase intensity of pain in the patient.

### b) Potential for Dependency

The μ-opioids used for treating pain, such as morphine and fentanyl, have a potential for dependency. In many cases, withdrawal symptoms occur when treatment with these medicaments is stopped. This side-effect of μ-opioids considerably limits the benefits of these highly active analgesics because, due to a fear of dependency, μ-opioids are often not prescribed or taken in cases of severe pain. There is therefore an urgent need for analgesics which are highly active and simultaneously exhibit a reduced potential for dependency in comparison with μ-opioids.

The typical side-effects of μ-opioids do not develop with equal strength in all patients. There are accordingly groups of patients for whom the side-effects are tolerable and others for whom they are a major problem. On average, however, the side-effects are a problem which it has not hitherto been possible to solve, despite μ-opioids, originally used as the naturally extracted substance opium, having long been used for treating pain. The first attempts to synthesize a morphine derivative without potential for dependency were made as long ago as 1874. It was found, however, that the resultant substance, heroin, did not have an improved side-effect profile in comparison with morphine. To date, numerous further attempts have been made to produce highly active analgesics with an improved side-effect profile. Oxycodone was accordingly synthesized in 1925, methadone in 1946, fentanyl in 1961 and tilidine in 1965. It has, however, been found that achieving a distinct reduction in side-effects is accompanied by a distinct reduction in efficacy. μ-Typical side-effects have been thoroughly investigated; they may be antagonized with the μ-antagonist naloxone and thus belong to the profile of action of μ-opioids. To date, there are no medicaments which have the same effectiveness as the clinically used step 3 μ-opioids (WHO ladder), such as fentanyl, sufentanil, morphine, oxycodone, buprenorphine and hydromorphone, and simultaneously have a significantly reduced side-effect profile.

To summarize, it may be concluded that the treatment of moderately severe to severe pain, of both acute and chronic type, is largely based on the use of μ-opioids, despite all their disadvantages. Above all, this is due to the elevated effectiveness of these compounds. The disadvantages are however so considerable that, due to a fear of the side-effects, many patients, due both to their own concerns and to reservations on the part of the doctor, do not receive the necessary treatment. There is thus an urgent need for novel analgesics which are based on a mode of action which, on the one hand, has the elevated efficacy of μ-opioids, while nevertheless reducing the disadvantages such as dependency, increased perception of pain, respiratory depression and reduced efficacy in chronic pain.

The object of the present invention was therefore to provide a mode of action for medicaments, wherein medicaments which act in accordance with this mode of action, on the one hand, have the elevated efficacy of μ-opioids, but exhibit the disadvantages, such as dependency, respiratory depression and reduced efficacy in chronic pain, to a lesser extent in comparison with μ-opioids.

Said object is achieved by the present invention.

The invention provides the use of mixed ORL1/μ-agonists, which exhibit an affinity for the μ-opioid receptor of at least 100 nM (Kᵢ value, human) and an affinity for the human ORL-1 receptor, wherein the ratio between the affinity for ORL1/μ defined as 1/[K_{i(ORL1)}/K_{i(μ)}] is between 0.1 and 30, for the treatment of pain. The Kᵢ values are determined on recombinant CHO cells which express the particular receptor.

The phrase “ORL1/μ defined as 1/[K_{i(ORL1)}/K_{i(μ)}]” is abbreviated to “ORL1/μ”. The phrase “at least 100 nM” means that the affinity is 100 nM or better (“better” means the Kᵢ value is lower than 100 nM, for example, 99.9 nM).

It has surprisingly been found that compounds which exhibit a ratio of ORL1/μ from 0.1 to 30 form a window within which, while the ORL-1 component does indeed bring about a distinct reduction in some μ-typical side-effects such as respiratory depression and dependency, the antiopioid action of this component does not yet prevent analgesic action against acute pain. In contrast with acute pain, in chronic pain states an analgesic synergistic action of the ORL1 component and μ component is even achieved, i.e. of the respective contributions made by the action of the compounds on an individual receptor to yield the overall effectiveness. In this manner, in compounds which exhibit a ratio of ORL1 to μ defined as 1/[K_{i(ORL1)}/K_{i(μ)}] from 0.1 to 30, distinctly increased efficacy is achieved which makes it possible to reduce the dose in comparison with acute pain in order to achieve a satisfactory action. The ratio of ORL1 to μ defined as 1/[K_{i(ORL1)}/K_{i(μ)}] is preferably 0.1 to 20. The compounds according to the invention may also comprise metabolites of a parent substance, wherein the metabolites may exhibit the properties according to the invention individually or as a mixture of metabolites in combination with the remaining quantity of the parent substance.

With regard to the efficacy of the compounds, it is important that the affinity of the compounds or the affinity of the metabolites for the μ-opioid receptor is at least 100 nM (Kᵢ value, human). This value is of the same order as highly active μ-opioids in clinical use such as hydrocodone (human μ-OR Kᵢ 76 nM), ketobemidone (human μ-OR Kᵢ 22 nM) and meptazinol (Kᵢ 150 nM human μ-OR). The affinity of the compounds for the μ-opioid receptor is preferably at least 50 nM.

The stated surprising characteristics of compounds with the characteristics according to the invention have been demonstrated by extensive animal testing. The compounds exhibit a tolerance range of ORL1/μ-proportions and demonstrate the exceptional position of the mixed ORL1/μ-agonists in the range according to the invention. The medicaments selected for carrying out comparative testing are those which are used today to treat severe pain. The reference substances B1-B6 comprise the μ-opioids fentanyl, sufentanil, morphine, oxycodone, buprenorphine and hydromorphone, which are all step 3 opioids according to the WHO analgesic ladder. These medicaments currently constitute the gold standard for the treatment of severe pain.

The ORL1 receptor is homologous to the μ, κ and δ opioid receptors and the amino acid sequence of the endogenous ligand, the nociceptin peptide, exhibits a strong similarity with those of known opioid peptides. Activation of the receptor, which is induced by nociceptin, gives rise, via coupling with G_{i/o} proteins, to inhibition of adenylate cyclase, inhibition of voltage-dependent calcium channels and activation of potassium channels (Meunier et al., Nature 377, 1995, pp. 532-535; Ronzoni et al., Exp. Opin. Ther Patents 2001, 11, 525-546).

After intracerebroventricular administration, the nociceptin peptide exhibits a pronociceptive and hyperalgesic activity in various animal models (Reinscheid et al., Science 270, 1995, pp. 792-794). These findings may be explained as inhibition of stress-induced analgesia (Mogil et al., Neuroscience 75, 1996, pp. 333-337).

On the other hand, it has also been possible to demonstrate an antinociceptive effect of nociceptin in various animal models after intrathecal administration (Abdulla and Smith, J. Neurosci., 18, 1998, pp. 9685-9694). Thus, depending on the site of action and physiological state of the organism, nociceptin has both antinociceptive and pronociceptive characteristics.

It is furthermore known that the endogenous ORL-1 ligand nociceptin exhibits an action against neuropathic pain. It has moreover been possible to demonstrate that nociceptin and morphine exhibit a synergistic action against neuropathic pain (Courteix et al., Pain 2004, 110, 236-245). However, when administered systemically, nociceptin alone is not active against acute pain (measured by the tail flick test). Pure ORL-1 agonists are therefore possibly suitable for treating neuropathic pain. However, if the pain to be treated occurs in mixed form or if the spontaneous pain typical in cases of neuropathic pain occurs, pure ORL-1 agonists are not sufficiently active according to the findings from animal experimentation.

Mixed ORL1/μ-agonists are already known from the literature, for example from EP 0997464 or WO 1999059997. However, these documents only disclose structures which are described as mixed ORL1/μ-agonists, but without any specific biological data being stated, and without disclosing that compounds in the affinity range according to the invention exhibit advantages. WO 2001039775 discloses mixed ORL1/μ-agonists and a general range, not specified in greater detail, in which compounds may have an ORL1 and μ-affinity, but without demonstrating any advantage of such compounds.

### BRIEF DESCRIPTION OF THE DRAWINGS

The invention will now be described in greater detail with reference to the drawings, wherein each figure is a graph with a depiction as indicated below:

FIG. 1: Analgesic efficacy against acute pain and against neuropathic pain, Chung model

FIG. 2: Comparison of analgesic efficacy against acute pain and against neuropathic pain, Bennett model

FIG. 3: Antagonization of antinociceptive effect by B11, Chung model 30 min (B11 dosages stated in mg/kg).

FIG. 4: Antagonization of antinociceptive effect by B11 or naloxone (Chung model 30 min)

FIG. 5: Separation of antinociceptive and antiallodynic effect in neuropathic animals, morphine

FIG. 5a: Separation of antinociceptive and antiallodynic effect in neuropathic animals, morphine

FIG. 6: Separation of antinociceptive and antiallodynic effect in neuropathic animals, A4

FIG. 6a: Separation of antinociceptive and antiallodynic effect in neuropathic animals, A4

FIG. 7: Morphine in naïve and neuropathic animals, comparison

FIG. 8: A4 in naive and neuropathic animals, comparison

FIG. 9: Inflammatory pain: single motor unit discharges in spinalized rats, comparison of naïve animals and carrageenan-pretreated animals, A4

FIG. 10: Inflammatory pain: single motor unit discharges in spinalized rats, comparison of naive animals and carrageenan-pretreated animals, A4

FIG. 11: Inflammatory pain: single motor unit discharges in spinalized rats, comparison of naive animals and carrageenan-pretreated animals, morphine

FIG. 11a: Inflammatory pain: single motor unit discharge in spinalized rats, comparison of naive animals and carrageenan-pretreated animals, morphine

FIG. 12: Rat CFA-induced hyperalgesia: determination of the antinociceptive (incl. anti-hyperalgesic) effect (time dependency: 1 h to 4 days after CFA administration)

FIG. 12a: Modification of antinociceptive (incl. anti-hyperalgesic) effect

FIG. 13: Comparison of semimaximal active dosages of mixed ORL1/μ-agonists and standard opioids after i.v. bolus administration in a rodent model of acute pain (tail flick, rat)

FIG. 14: Occurrence of transient hyperalgesia after administration of fentanyl

FIG. 14a: Occurrence of transient hyperalgesia after administration of morphine

FIG. 14b: Occurrence of transient hyperalgesia after administration of A7

FIG. 14c: Occurrence of transient hyperalgesia after administration of A4

FIG. 15: Withdrawal jumping after administration of levomethadone

FIG. 15a: Withdrawal jumping after administration of B8

FIG. 15b: Withdrawal jumping after administration of A1

FIG. 15c: Withdrawal jumping after administration of A9

FIG. 15d: Withdrawal jumping after administration of A4 with morphine as comparison substance

FIG. 15e: Withdrawal jumping after administration of A7 with morphine as comparison substance

FIG. 16: Spontaneous withdrawal

FIG. 17: Time profile of analgesic action of fentanyl in the tail flick test and, by way of comparison, the time profile of arterial pCO₂ in each case for a fully analgesically active dosage and the analgesic threshold dosage (administration in each case as i.v. bolus)

FIG. 17a: Time profile of analgesic action of oxycodone in the tail flick test and, by way of comparison, the time profile of arterial pCO₂ in each case for a fully analgesically active dosage and the analgesic threshold dosage (administration in each case as i.v. bolus).

FIG. 17b: Time profile of analgesic action of A4 in the tail flick test and, by way of comparison, the time profile of arterial pCO₂ in each case for a fully analgesically active dosage and the analgesic threshold dosage (administration in each case as i.v. bolus).

FIG. 17c: Time profile of analgesic action of A5 in the tail flick test and, by way of comparison, the time profile of arterial pCO₂ in each case for a fully analgesically active dosage and the analgesic threshold dosage (administration in each case as i.v. bolus).

FIG. 17d: Time profile of analgesic action of A6 in the tail flick test and, by way of comparison, the time profile of arterial pCO₂ in each case for a fully analgesically active dosage and the analgesic threshold dosage (administration in each case as i.v. bolus).

FIG. 17e: Time profile of analgesic action of A9 in the tail flick test and, by way of comparison, the time profile of arterial pCO₂ in each case for a fully analgesically active dosage and the analgesic threshold dosage (administration in each case as i.v. bolus).

FIG. 18: Detection of the positive effect of mixed ORL1/μ-agonists on respiratory depression with reference to antagonization experiments

FIG. 19: Margins between analgesia and side-effect taking comparative respiratory depression for pure μ-opioids and mixed ORL1/μ-agonists by way of example

FIG. 20: Margins between analgesia and side-effect taking psychological dependency for pure μ-opioids and mixed ORL1/μ-agonists by way of example

FIG. 21: Observed place preference after administration of A7

FIG. 22: Enhancement of place preference after antagonization of the ORL1 component

FIG. 23: Cytostatic-induced polyneuropathy pain, A4

FIG. 24: Cytostatic-induced polyneuropathy pain, morphine

FIG. 25: STZ-induced polyneuropathy pain, A4, three dosages [a), b) & c)]

FIG. 26: STZ-induced polyneuropathy pain, morphine, two dosages [a) & b)]

FIG. 27: STZ-induced polyneuropathy pain, pregabalin, three dosages [a), b) & c)]

### ENHANCEMENT OF ACTION AGAINST CHRONIC PAIN IN COMPARISON WITH PURE μ-OPIOIDS

### a) Neuropathic Pain

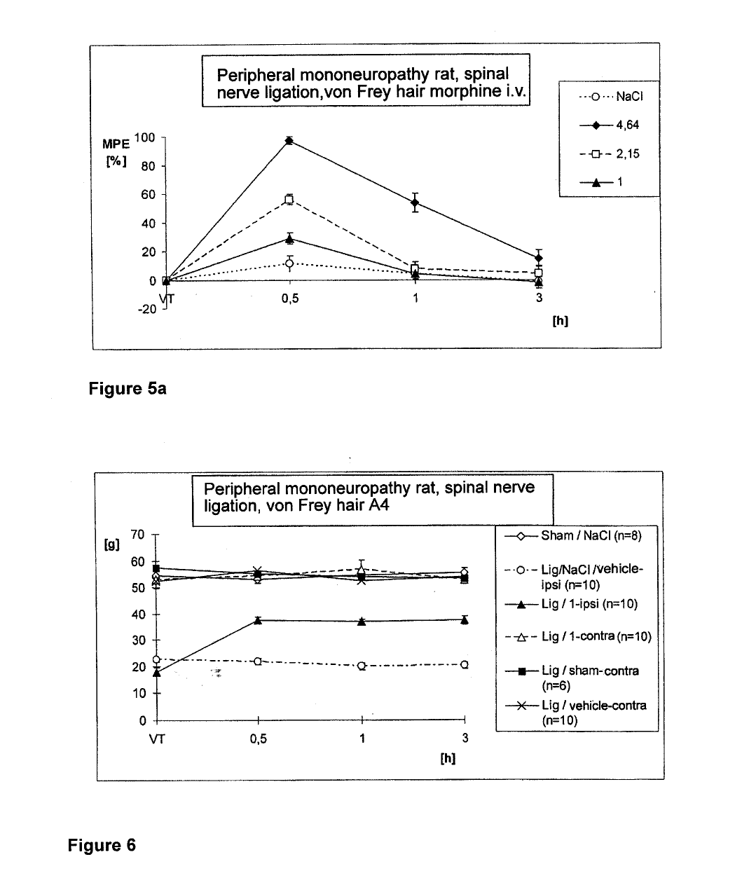

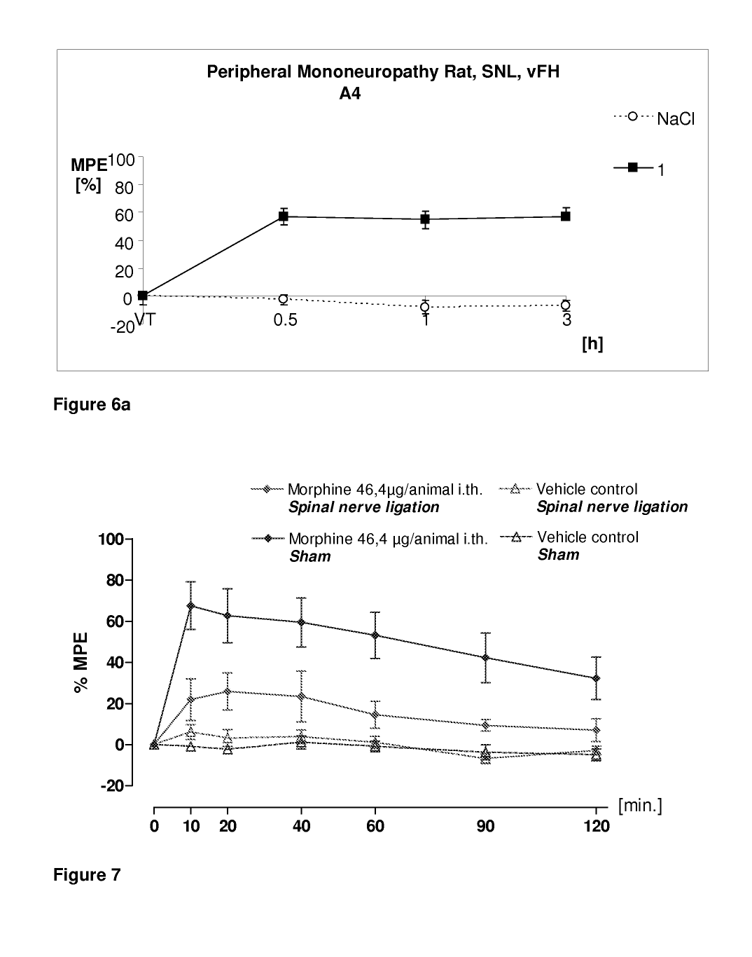

In models of neuropathic pain, it is surprisingly possible in the case of mixed ORL1/μ-agonists, in contrast with conventional μ-agonists, to observe a distinct increase in analgesic efficacy in the range from 0.1 to 30, preferably of up to 20. It has been shown in antagonization experiments that the ORL1 component in mixed ORL1/μ-agonists makes a direct contribution to analgesic action (FIG. 3). Direct comparison of a substance with an ORL1/μ-ratio of 0.5 (compound A4) and morphine in naive and neuropathic animals shows that, once neuropathy has developed, the efficacy of morphine declines (which corresponds to the clinical situation), whereas it has a tendency to increase for the mixed agonists (FIGS. 5, 5a, 6, 6a).

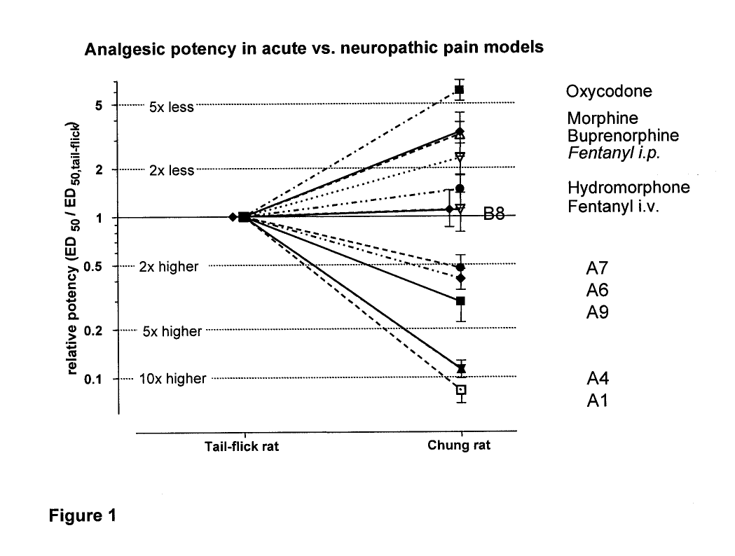

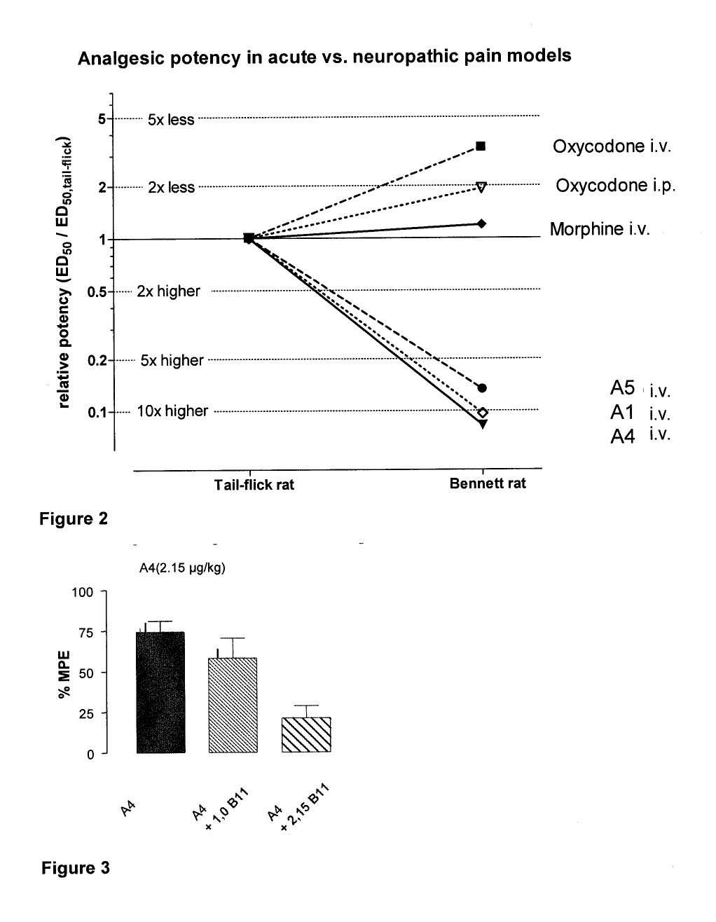

Comparison of analgesic efficacy in the acute pain model (tail flick, rat/mouse) and in neuropathic pain models, the Chung model in rats and the Bennett model in rats/mice, reveals the exceptional position of the compounds with the characteristics according to the invention (see FIGS. 1 and 2). In contrast with pure μ-opioids, in which analgesic potency in the neuropathic pain model is lower than in the acute pain model (by up to a factor of 5), the analgesic potency of mixed ORL1/μ-agonists in the neuropathic pain model is higher by a factor of 2 to 10 than in the acute pain model. Accordingly, the clinically used μ-opioid oxycodone is, for example, three to five times less potent against neuropathic pain in comparison with acute pain (depending on the animal model); a mixed agonist with an ORL1/μ-ratio of 0.5 (compound A4), in contrast, is approximately ten times more potent against neuropathic pain than against acute pain.

The upper limit of the range within which the effect occurs is demonstrated by the compound B8, which exhibits an ORL1/μ-ratio of 0.03 and is no longer any more active in the neuropathic model than it is in the acute pain model. Example A1 (ORL1/μ-ratio 0.1), in contrast, is still more active by a factor of 10.

Compound A11 with an ORL1/μ-ratio of 20, when administered intrathecally, exhibits still greater enhancement of action against neuropathic pain. When administered systemically, the compound still remains highly active against acute pain (tail flick mouse i.v. ED₅₀=0.42 mg/kg). Compound B9 with an ORL1/μ-ratio of 140:1, when administered intrathecally, likewise exhibits a great enhancement of action against neuropathic pain. When administered systemically, the compound is, however, no longer active against acute pain due to the excessively low μ component. The endogenous ORL-1 ligand nociceptin no longer exhibits any action in the acute pain model (tail flick i.v.). Due to the antiopioid characteristics of the ORL1 component, in those compounds which have an ORL1 component which is present in an amount distinctly better than 30:1 in comparison with the μ component, action against acute pain is too poor to be comparable with the effectiveness of a step 3 opioid. This relationship may be explained by the antagonization of the ORL1 component. The findings show that the compounds with the characteristics according to the invention constitute a defined subgroup of mixed μ/ORL1 agonists which have the disclosed extraordinary characteristics. The lower limit of the range according to the invention is therefore at 30, preferably at 20.

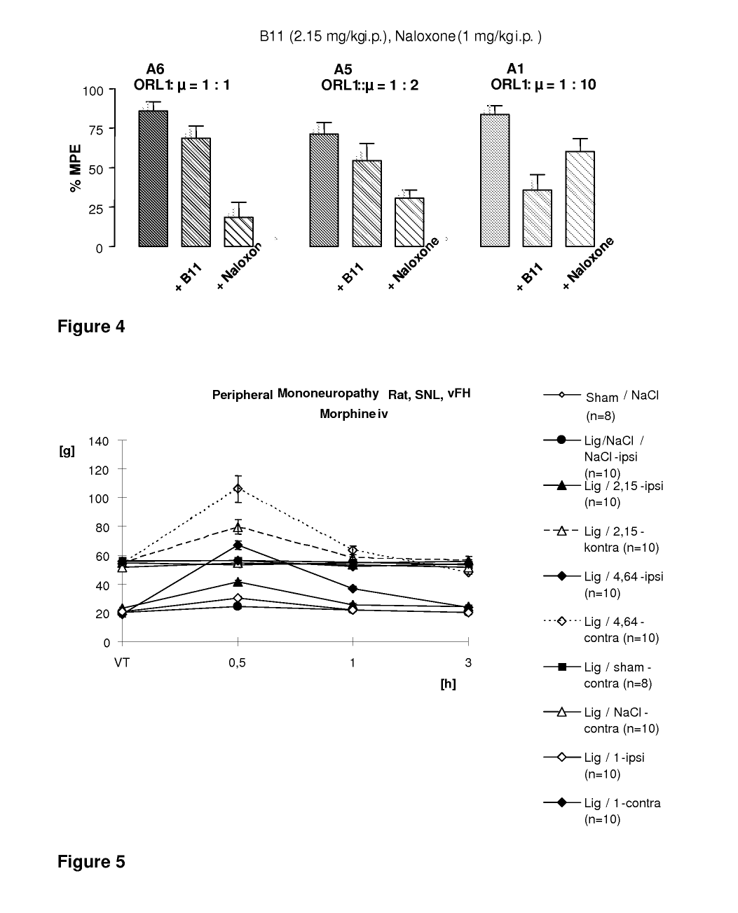

It has been demonstrated in antagonization experiments using the Chung model that the analgesic efficacy of the mixed ORL1/μ-agonists is based on both components. After administration of mixed ORL1/μ-agonists, partial nullification of the analgesic action can be demonstrated both with a μ-antagonist and with an ORL1 antagonist (FIGS. 3 and 4). This confirms that both the μ-opioid component and the ORL1 component contribute towards the action against chronic neuropathic pain.

The antagonization experiments clearly show that the characteristics according to the invention are directly attributable to the ORL1 agonistic and the μ-agonistic action of the compounds.

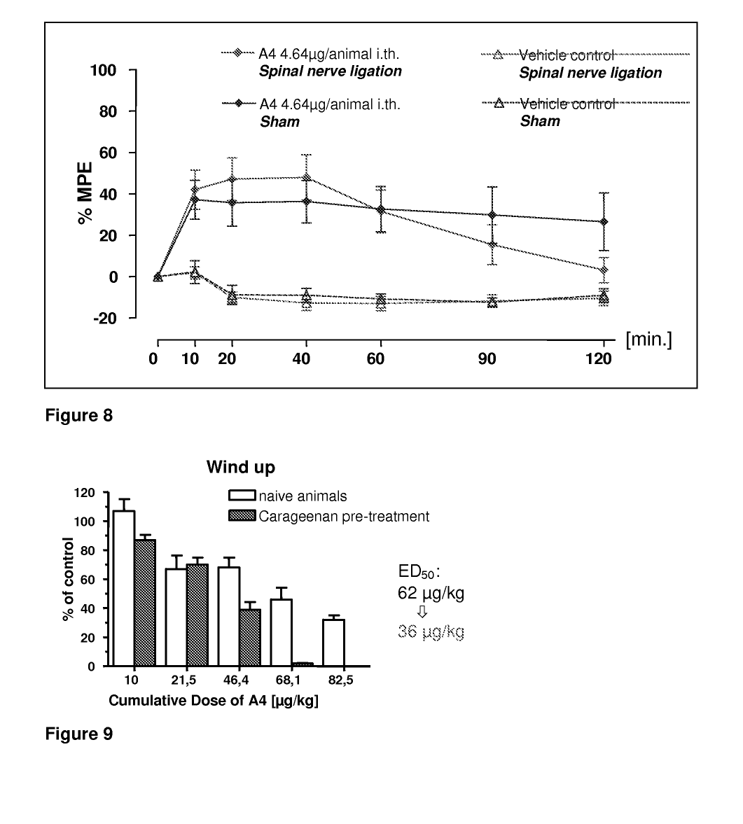

In order to exclude a possible influence of “pain quality” (tail flick, nociceptive stimulus vs. Chung, tactile allodynia) in the comparison of differing efficacy against acute pain and neuropathic pain, A4 and morphine were subjected to comparative testing in Chung animals and sham operated animals. In every case, the pain model used was the tail flick. The direct comparison shows that, while morphine does indeed exhibit a very good action on sham operated animals (which corresponds to the situation against acute pain), once neuropathy has developed in operated animals, the efficacy of morphine is comparatively distinctly less (FIG. 7). This corresponds to the clinical situation and demonstrates one of the problems of μ-opioids in clinical practice. A4 on the other hand exhibits a clear action on sham operated animals, which even increases further once neuropathy has developed (FIG. 8). This shows the distinct advantage of mixed ORL1/μ-agonists in comparison with pure μ-opioids in the treatment of neuropathic pain.

The compounds with an ORL1/μ-ratio defined as 1/[K_{i(ORL1)}/K_{i(μ)}] of 0.1 to 30, preferably of 0.1 to 20, at a Kᵢ value on the μ-opioid receptor of below 100 nM are thus preferably used for the treatment of neuropathic pain.

Separation of Antinociceptive and Antiallodynic Effect in Neuropathic Animals Another advantage of the mixed ORL1/μ-agonists in the range according to the invention is the separation of the antinociceptive and antiallodynic effects. In allodynia, pain is evoked by a stimulus which is certainly not painful on an unaffected part of the body (e.g. touch, heat or cold stimulus). Mechanical allodynia is typical in postzoster neuralgia, while cold allodynia is frequent in posttraumatic nerve lesions and some types of polyneuropathy. Mechanical allodynia in particular typically occurs in diabetic neuropathy (Calcutt and Chaplan, Br. J. Pharmacol. 1997, 122, 1478-1482).

In certain groups of chronic pain patients, it is advantageous for allodynia and hyperalgesia to be combated while leaving normal pain perception largely in place. These patients, for whom it is sensible to have the protective mechanism of pain in their daily life, therefore require medication which combats specifically only allodynia and hyperalgesia, but leaves general pain perception as unaffected as possible. This applies, for example, to postzoster neuralgia, with which pain is typically induced by stimuli which are usually not painful at all, such as e.g. by gentle contact or clothing.

In the Chung model, it is possible by comparative testing of the pain response on the ipsilateral and contralateral paw (relative to the side on which the spinal nerve ligature has been placed) to distinguish between antinociceptive (contralateral) and antiallodynic (ipsilateral) action.

In the case of the μ-agonist morphine, a purely antiallodynic action could be observed only once 1 mg/kg i.v. had been administered. Maximum efficacy here amounts to 29% MPE (maximum possible effect), which amounts to a perceptible, but weak action. Onset of a distinct antinociceptive action is already observed at the next highest test dose (2.15 mg/kg i.v.) (FIGS. 5, 5a). It is thus not possible to achieve a clear separation between a distinct antiallodynic effect and an antinociceptive effect with morphine.

In contrast, the maximum, purely antiallodynic action of A4 is 56% MPE. This is achieved at a test dose of 1 μg/kg i.v. and corresponds to good level of efficacy (FIGS. 6, 6a). This demonstrates another advantage of the mixed ORL1/μ-agonists in comparison with pure μ-opioids.

It is therefore also preferred to use the compounds with an ORL1/μ-ratio of 0.1 to 30, preferably of 1:10 to 20:1, at a Kᵢ value on the μ-opioid receptor of below 100 nM for the treatment of allodynia, hyperalgesia and spontaneous pain, preferably at a dosage at which the general perception of pain is largely retained. Retention of the general perception of pain in humans may be verified using the cold pressor model (Enggaard et al., Pain 2001, 92, 277-282).

It is furthermore preferred to use the compounds with an ORL1/μ ratio of 0.1 to 30, preferably of 0.1 to 20, at a Kᵢ value on the μ-opioid receptor of below 100 nM for the treatment of pain with postzoster neuralgia.

For more detailed study of the diverse forms of neuropathy pain, A4 was investigated in a model for investigation of cytostatic-induced polyneuropathy pain. Cytostatic-induced polyneuropathy pain is a highly clinically relevant sub-group of neuropathy pain. Polyneuropathy was induced by administration of the cytostatic vincristine. A syndrome which mirrors the clinical situation following chemotherapy with vincristine thus developed in the rat. Morphine was investigated here as a comparison substance.

A4 showed a significant efficacy from a dosage of 1 μg/kg, i.e. from a dosage which lies in the ED₅₀ region against chronic pain. At the lower dosage of 0.464 μg/kg, however, no significant efficacy was yet to be seen (FIG. 23). For morphine, a good efficacy is observed from a dosage of 2.15 mg/kg (ED₅₀ Chung rat 3.7 mg/kg).

The efficacy against diabetes-induced polyneuropathy pain was furthermore investigated. This form of pain was investigated in a model on the rat, diabetic polyneuropathy being induced by administration of streptozotocin. A4 already showed a significant inhibition of diabetes-induced mechanical hyperalgesia in the rat at the lowest dosage tested of 0.316 μg/kg i.v., and therefore in a lower dose range than in the case of cytostatic-induced polyneuropathy pain, with which no significant efficacy was yet to be observed at a dosage of 0.464μ/kg.

In this low dose range A4 had no effect on the control group. This means that against diabetes-induced polyneuropathy pain

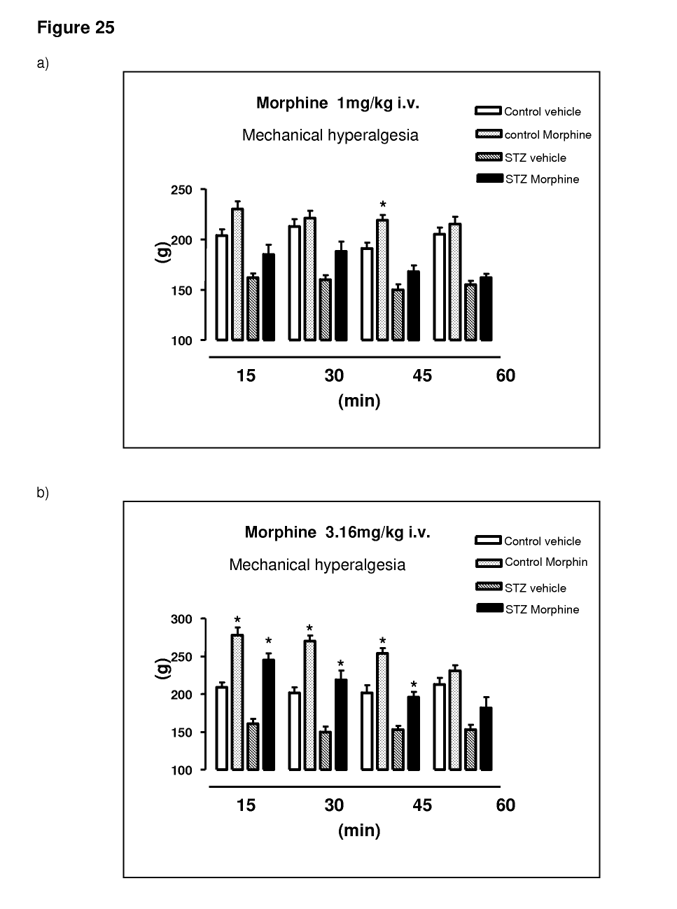

1.) surprisingly the efficacy of A4 is one more better than against other forms of neuropathy pain and
2.) the anti-hyperalgesic action of A4 already exists in a dose range in which no anti-nociceptive action yet emerges (FIG. 25), and therefore alleviation of polyneuropathy pain is possible, without the sensation of acute pain being impaired.

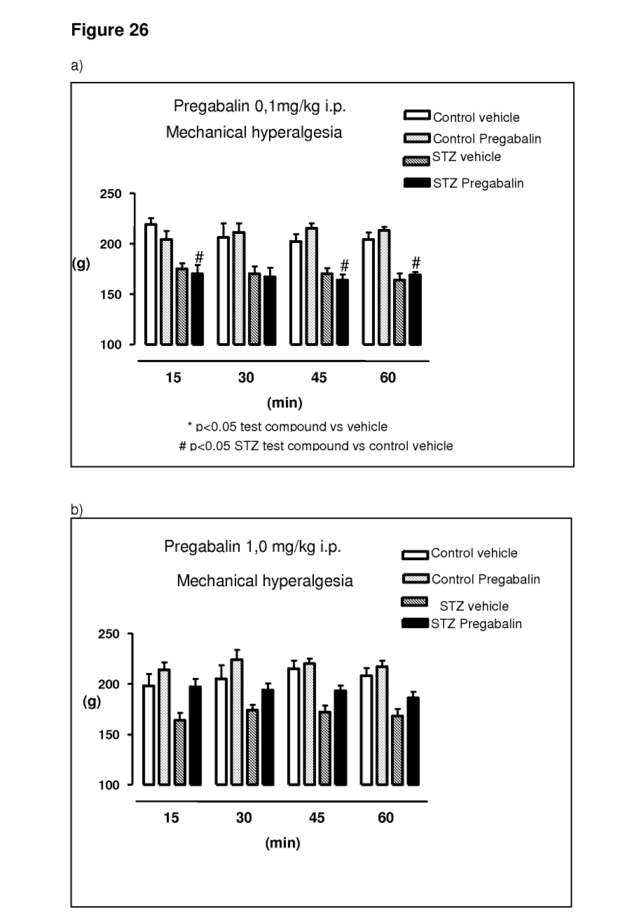

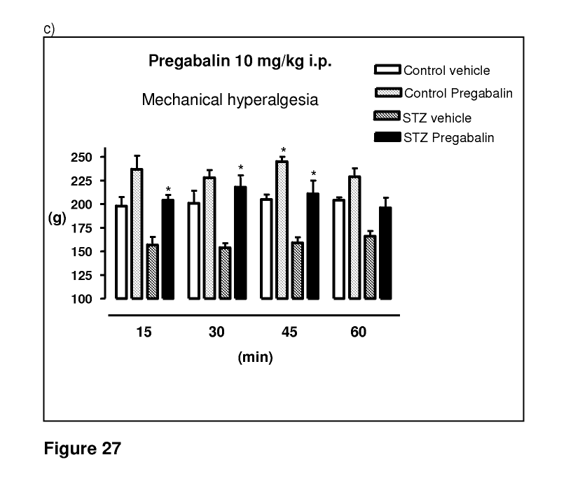

With morphine, on the other hand, an anti-hyperalgesic action is to be observed only in a dose range in which an antinociceptive action also emerges in the control group (FIG. 26). Since the standard therapy against diabetes-induced polyneuropathy pain is currently not administration of a μ-agonist such as morphine, but, inter alia, administration of pregabalin, pregabalin was investigated in the same model as a further comparison. Here also it was found that an anti-hyperalgesic action is first to be observed in a dose range in which an antinociceptive action also emerges in the control group (FIG. 27). This underlines the exceptional efficacy of the compounds with the properties according to the invention against diabetes-induced polyneuropathy pain.

Compounds with an ORL1/μ ratio of 0.1 to 30, preferably of 0.1 to 20, at a Kᵢ value on the μ-opioid receptor of below 100 nM are therefore particularly preferably used for the treatment of diabetic polyneuropathy pain.

### b) Inflammatory Pain

It proved possible to demonstrate in two in vivo models (single motor unit discharge on spinalized rats and CFA**-induced hyperalgesia) that the efficacy of mixed ORL1/μ-agonists is increased after chronic inflammation.

Single Motor Unit Discharges in Spinalized Rats. Comparison of Naive Animals and Animals after Carrageenan-Induced Inflammation.

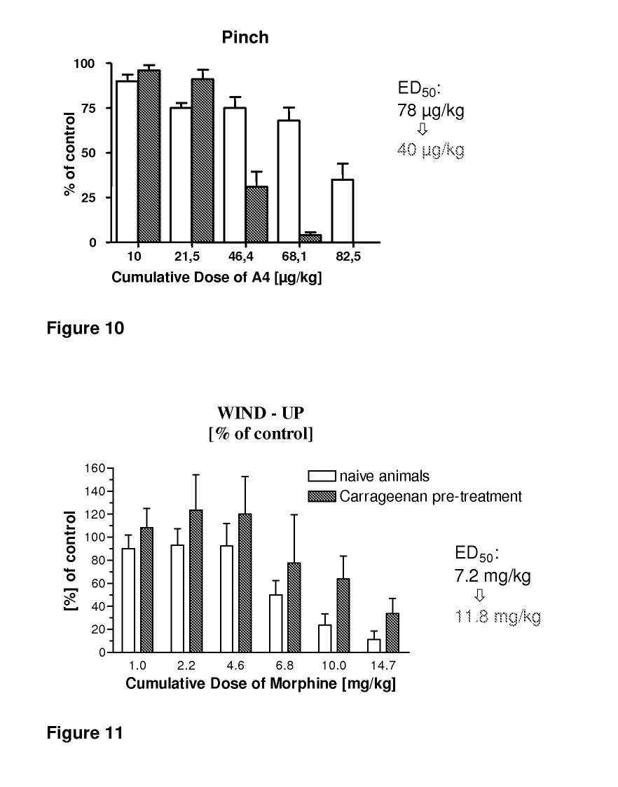

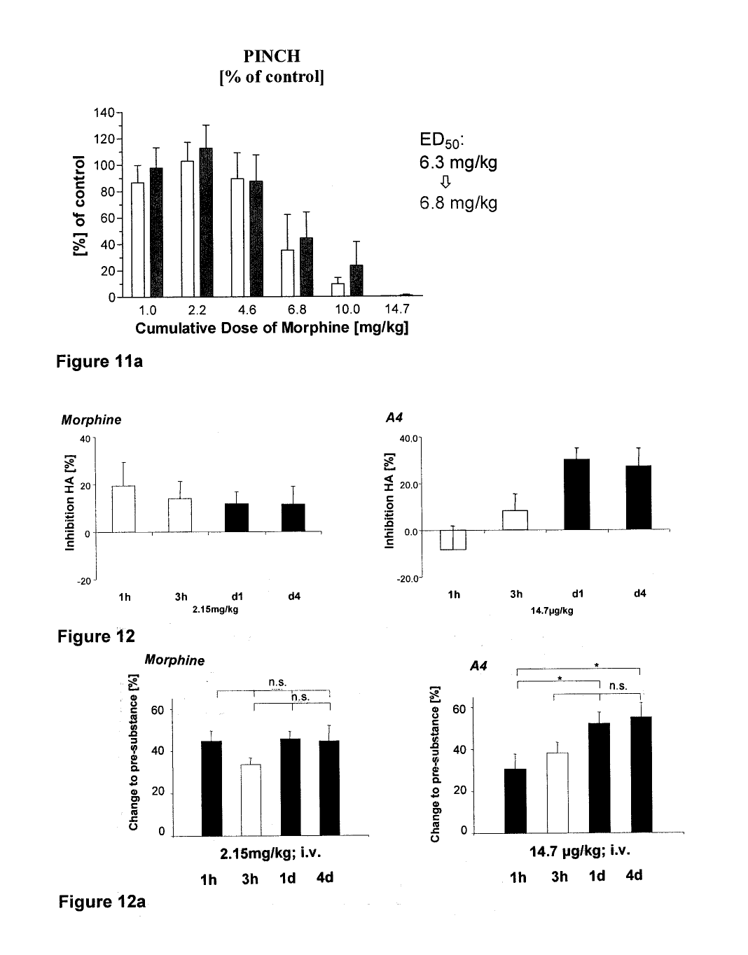

It has been observed in rats that the antinociceptive action of A4 (ORL1/μ ratio 1:2, FIGS. 9 and 10) and A11 (ORL1:μ ratio 20:1) is distinctly increased 24 h after induction of inflammation compared with the value before the inflammation. The antinociceptive action of the μ-agonist morphine, in contrast, tends to be weaker after inflammation (FIGS. 11 and 11a). This shows that, after chronic inflammation, the efficacy of mixed ORL1/μ-agonists is increased, while that of pure μ-agonists is not.

### CFA-Induced Hyperalgesia

In a model of chronic inflammatory pain, inflammation was induced in the hind paw by injecting CFA. Tactile hyperalgesia and nociception were determined 1 h, 3 h, 24 h and 4 days after induction of inflammation. While morphine exhibited a slightly declining antihyperalgesic action and an unchanging antinociceptive action over the entire investigation period, the antihyperalgesic and antinociceptive action of A4 increased over 24 h. The effect is stable for at least 4 days (FIGS. 12 and 12a). This shows that, in a similar manner to the situation with neuropathic pain, mixed ORL1/μ-agonists are distinguished by a distinct enhancement of action in inflammatory pain relative to analgesia in acute pain.

### Visceral Inflammatory Pain

Comparative testing of A4 and fentanyl in a model of transferred allodynia and transferred hyperalgesia in mice after non-neurogenic visceral inflammation induced by mustard oil revealed significantly higher efficacy of the mixed ORL1/μ-agonists for both pain parameters in comparison with the pure μ-opioid. The analgesic efficacy of A4 in relation to both the tested pain parameters is higher by a factor of approx. 6 to 7 than against acute pain. In contrast, the analgesic efficacy of fentanyl against visceral inflammatory pain is lower than against acute pain. This likewise shows that, in a similar manner to the situation with neuropathic pain, mixed ORL1/μ-agonists are distinguished by a distinct enhancement of analgesic action in visceral inflammatory pain relative to acute pain. In addition to the reduced side effects in comparison with pure μ-opioids, the compounds therefore also show a better efficacy against inflammatory pain.

Mixed ORL1/μ-agonists with an ORL1:μ ratio of 0.1 to 30, preferably of 0.1 to 20, at a Kᵢ value on the μ-opioid receptor of below 100 nM are accordingly distinguished by a high efficacy against inflammatory pain. The invention therefore also provides the use of compounds with an ORL1:μ ratio of 0.1 to 30, preferably of 0.1 to 20, at a Kᵢ value on the μ-opioid receptor of below 100 nM for the treatment of patients suffering from inflammatory pain. The inflammatory pain can be induced, for example, by rheumatoid arthritis or pancreatitis.

It has been shown that mixed ORL1/μ-agonists with an ORL1:μ-ratio of 0.1 to 30, preferably of 0.1 to 20, at a Kᵢ value on the μ-opioid receptor of below 100 nM exhibit enhanced action against chronic pain in comparison with acute pain. It is therefore preferred to use the compounds against chronic pain at a dosage which is below the dosage which is necessary against acute pain. The compounds are preferably used against chronic pain at a dosage which is lower by a factor of at least 2 than the dosage used against acute pain, particularly preferably lower by a factor of at least 5. In animals, the dosage may be determined as the ED₅₀ value in the tail flick test, in humans by the cold pressor model (Enggaard et al., Pain 2001, 92, 277-282).

### c) Acute Pain

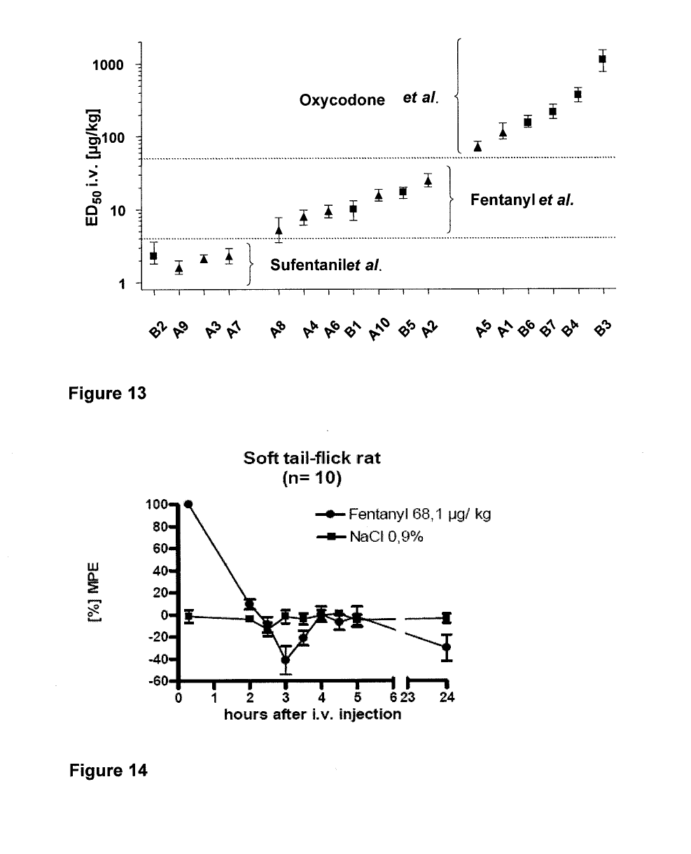

The mixed ORL1/μ-agonists with an ORL1:μ-ratio of 0.1 to 30, preferably of 0.1 to 20, exhibit full efficacy in various acute pain models and species after i.v. administration. It has proved possible to demonstrate this effect both in rats and in mice (tail flick, FIG. 13).

In a comparison of mixed ORL1/μ-agonists with pure μ-agonists, the mixed ORL1/μ-agonists exhibit comparable efficacy combined with better compatibility. These results show that the mixed ORL1/μ-agonists also exhibit excellent efficacy against acute pain. In their efficacy against acute pain, the compounds are comparable with step 3 opioids. The means that these are compounds which exert their analgesic action by a mechanism differing from that of pure μ-antagonists, which have for centuries dominated the treatment of severe pain, but have the same effectiveness. Apart from their surprising enhancement of action against chronic pain in comparison with acute pain, compounds with the binding profile according to the invention also exhibit a distinctly improved side-effect profile in comparison with pure μ-agonists.

### d) Side-Effects

### Opioid-Induced Hyperalgesia

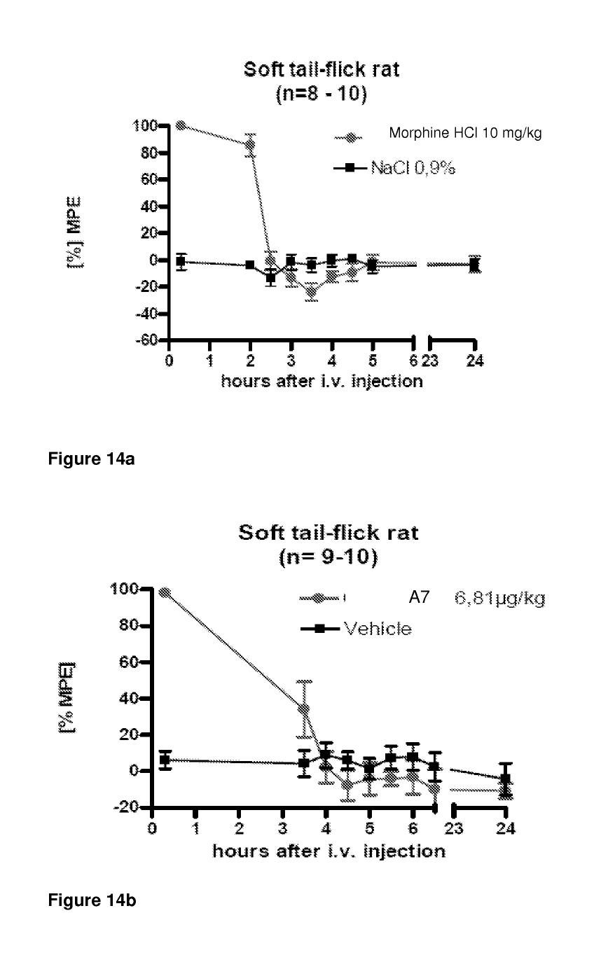

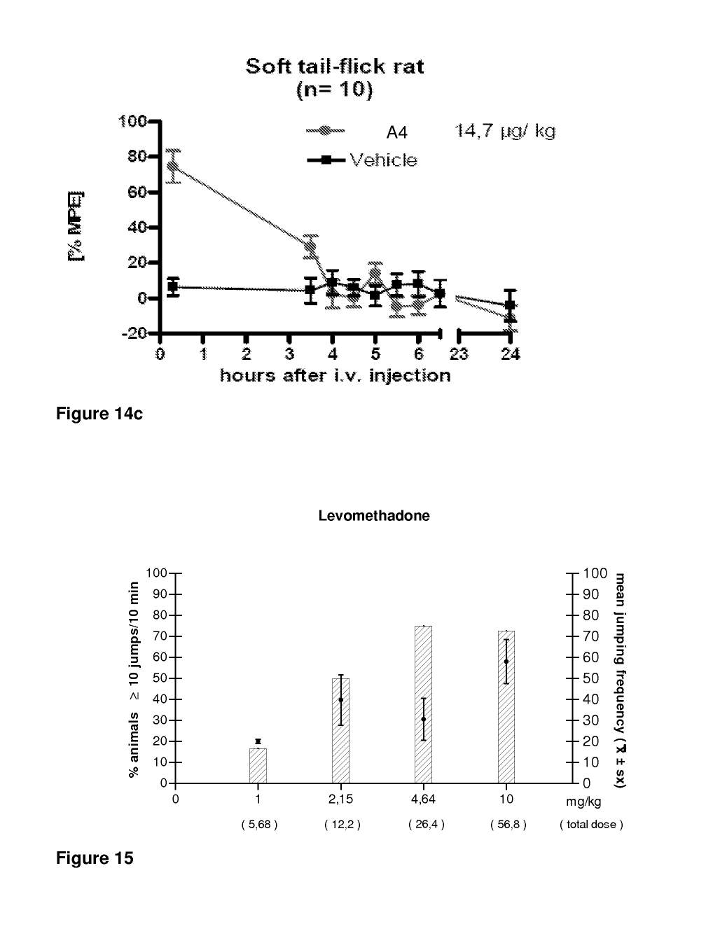

Chronic administration of opioids leads to hyperalgesia in pain patients (cf. Chu et al. 2006, J. Pain 7:43-48). A similar phenomenon also occurs after acute administration in the withdrawal situation (Angst et al. 2003, Pain 106: 49-57). In an animal model, pure μ-opioids induce transient hyperalgesia after acute administration, which may be detected, for example, in the soft tail flick model as a transient “pronociceptive” phase. This opioid-induced hyperalgesia may be demonstrated with the assistance of a modified tail flick model using a reduced strength of stimulus (25% intensity of thermal radiation) for pure μ-opioids (fentanyl and morphine). In contrast, no transient hyperalgesia was observed after acute administration of mixed ORL1/μ-agonists (A4 and A7) (FIGS. 14-14c).

This shows that chronic administration of a mixed ORL1/μ-agonist does not induce hyperalgesia or induces hyperalgesia which is reduced relative to pure μ-opioids. One of the typical side-effects of μ-opioids is accordingly reduced in mixed ORL1/μ-agonists.

The compounds with an ORL1/μ-ratio of 0.1 to 30, preferably of 0.1 to 20, at a Kᵢ value on the μ-opioid receptor of below 100 nM are thus preferably used to reduce opioid-induced hyperalgesia in the treatment of pain.

The use of the compounds with an ORL1/μ ratio of 0.1 to 30, preferably of 0.1 to 20, at a Kᵢ value on the μ-opioid receptor of below 100 nm for the treatment of patients who have an increased risk of developing hyperalgesia is particularly advantageous. These include, for example, patients who are already suffering from hyperalgesia and have to undergo an operation, such as, for example, irritable colon patients (visceral hyperalgesia), tumor pain patients and patients with musculoskeletal pain or patients who have received intraoperatively a potent opioid, such as fentanyl, intrathecally (e.g. Caesarean section patients). The invention therefore also provides the use of compounds with an ORL1/μ ratio of 0.1 to 30, preferably of 0.1 to 20, at a Kᵢ value on the μ-opioid receptor of below 100 nM for alleviation of pain in patients who have an increased risk of developing hyperalgesia.

The invention also provides the use of compounds which exhibit an affinity of at least 100 nM for the μ-opioid receptor and for the ORL1 receptor and, due to the ORL1 component, induce hyperalgesia which is reduced in comparison with a μ-opioid of the same affinity range, for the treatment of pain.

### Withdrawal

In naloxone-induced withdrawal jumping in mice, it proved possible to show that withdrawal jumping is suppressed by compounds with an ORL-1 component which is less than a factor of 10 weaker than the μ component. Compounds with a weaker ORL1 component, in contrast, trigger withdrawal jumping. In the “withdrawal jumping” test, mice are treated repeatedly with the test substance over a defined period. In the case of a μ-opioid, physical dependency is achieved within this period. At the end of the treatment, the action of the opioid is abruptly nullified by administering naloxone, a μ-antagonist. Where physical dependency has developed, the mice exhibit characteristic withdrawal symptoms which are manifested in the form of jumping movements (Saelens J K, Arch. Int. Pharmacodyn. 190: 213-218, 1971).

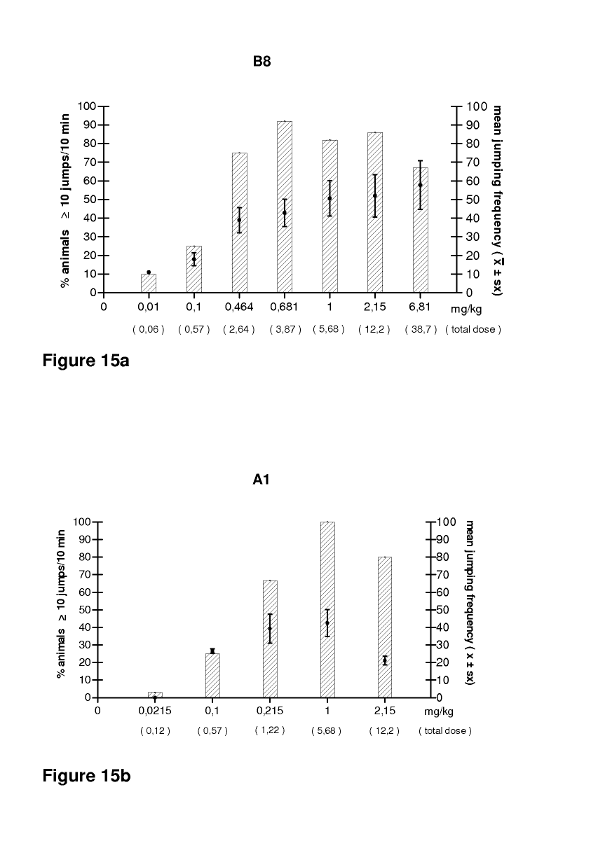

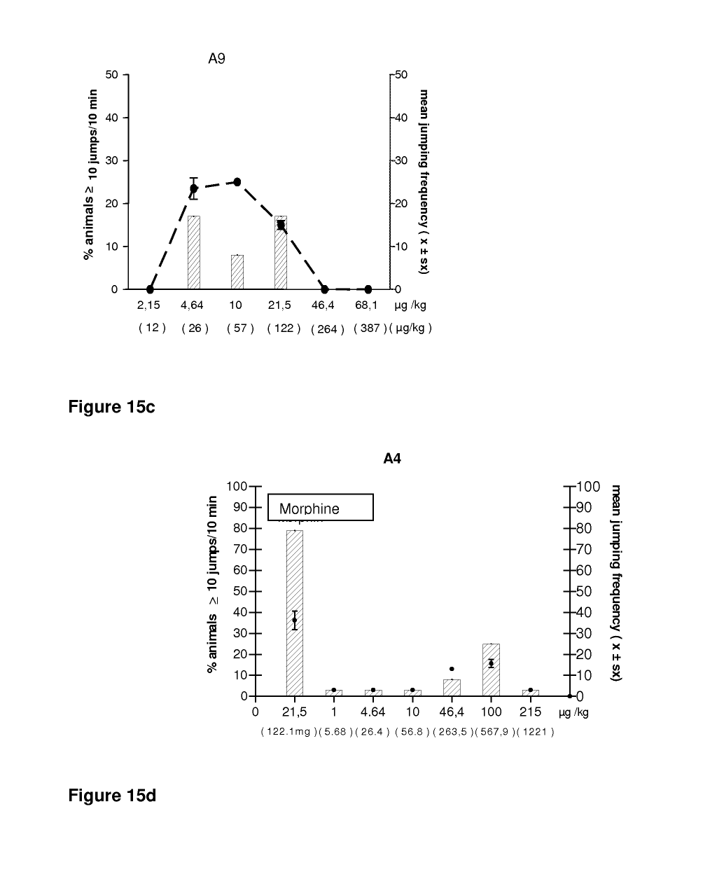

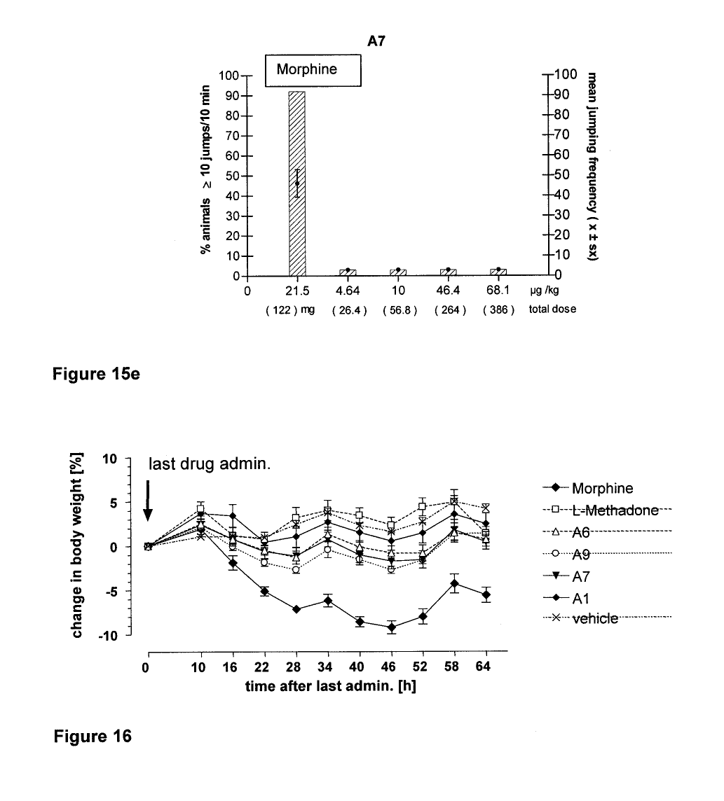

The compounds with the characteristics according to the invention have, thanks to the ORL1 active component, additional characteristics which pure μ-opioids do not have and enhance therapy. It has been shown by withdrawal jumping in mice that, in those animals which have been treated with combined ORL1/μ-agonists such as A9, A6, A4 or A7, naloxone triggers no or only minimal withdrawal behavior (see FIGS. 15c-e). A1, in contrast, does exhibit distinct withdrawal symptoms in terms of withdrawal jumping (FIG. 15b). In spontaneous withdrawal in rats, in which the weight of the rat is documented over several days after stopping treatment with the test substance, there is, however, a distinct difference to be found between morphine and A1 (ORL1:μ 0.1) (FIG. 16). While the weight of the rats falls by almost 10% after stopping treatment with morphine, it only falls by 3% after stopping treatment with A9. Here too, the ORL1/μ ratio of 0.1 is a limit up to which the advantageous action of the compounds with the characteristics according to the invention is to be observed. Thanks to these characteristics, the compounds with an ORL1/μ-ratio of 0.1 to 30, preferably of 0.1 to 20, at a Kᵢ value on the μ-opioid receptor of below 100 nM are particularly suitable for patient groups who have an increased risk of physical dependency. This group may, for example, include patients who already have experience of μ-opioids.

However, for the purpose of suppressing physical dependency, it is preferred for the ORL1 component to be somewhat increased, wherein physical dependency is however already reduced at an ORL1:μ-ratio of 0.1. The ORL1/μ ratio of a compound for the treatment of pain with the simultaneous suppression of withdrawal symptoms preferably amounts to at least 0.25, particularly preferably at least 0.5. Compounds with this increased ORL1 component are preferably used in patient groups who have a particular risk of physical dependency.

The invention also provides the use of compounds which exhibit an affinity of at least 100 nM for the μ-opioid receptor and for the ORL1 receptor and, due to the ORL1 component, induce withdrawal symptoms which are reduced in comparison with a μ-opioid of the same affinity range, for the treatment of pain. The effect may be demonstrated by the models relating to withdrawal jumping and to spontaneous withdrawal described in the Examples.

### Reduction of Psychological Dependency/Addiction

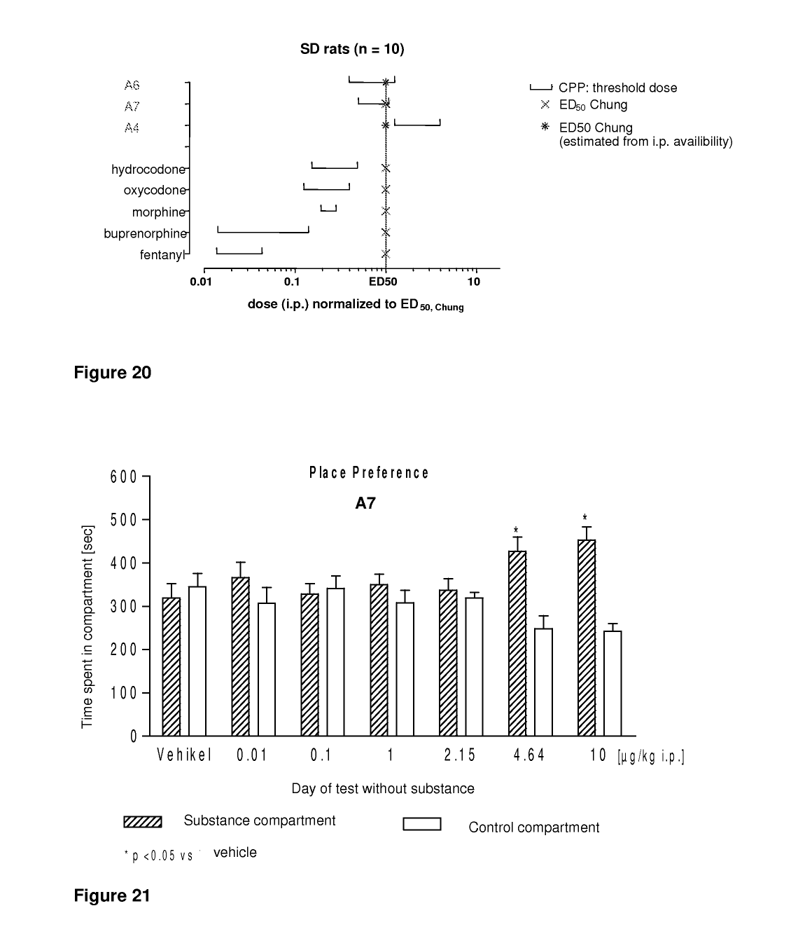

Mixed ORL1/μ-agonists induce, in a similar manner to pure μ-agonists, place conditioning in rats. While the threshold dose for inducing a place preference with pure μ-opioids (for example B1, B3-B6) is distinctly below the analgesically semimaximal active dose, with mixed ORL1/μ-agonists (for example A4, A7 and A6) it is in the range of or above the analgesically semimaximal active dose (FIG. 20). This means that mixed ORL1/μ-agonists exhibit an addictive potential which is reduced relative to pure μ-opioids.

Despite their potential for physical and psychological dependency, μ-opioids have long successfully been used in clinical practice, with most patients ceasing to take the medicament once treatment is complete. However, certain patient groups are susceptible to addictive behavior. It is therefore preferred to use the compounds with the characteristics according to the invention for treating pain in patients having an elevated potential for addiction.

These patient groups for example include people with psychological disorders, in particular depressive people or people suffering from anxiety disorders (Paton et al., Journal of Genetic Psychology 1977, 131, 267-289). The compounds with the characteristics according to the invention are therefore preferably used in patients exhibiting a psychological complaint in order to avoid the hazard of psychological dependency in the course of the pain therapy. The compounds with the characteristics according to the invention are particularly preferably used for pain therapy in patients suffering from depression or anxiety disorders.

The invention also provides the use of compounds which exhibit an affinity of at least 100 nM for the μ-opioid receptor and for the ORL1 receptor and, due to the ORL1 component, bring about psychological dependency which is reduced in comparison with a μ-opioid of the same affinity range, for the treatment of pain. This effect may, for example, be demonstrated by antagonization experiments, but also by place preference investigations, as described in the Examples.

### Respiratory Depression

μ-Mediated respiratory depression is distinctly reduced in mixed ORL1/μ-agonists. Acute respiratory depressive action was measured as the increase in pCO₂ of the arterial blood in rats both at an analgesically fully effective dose and at a threshold analgesic dosage.

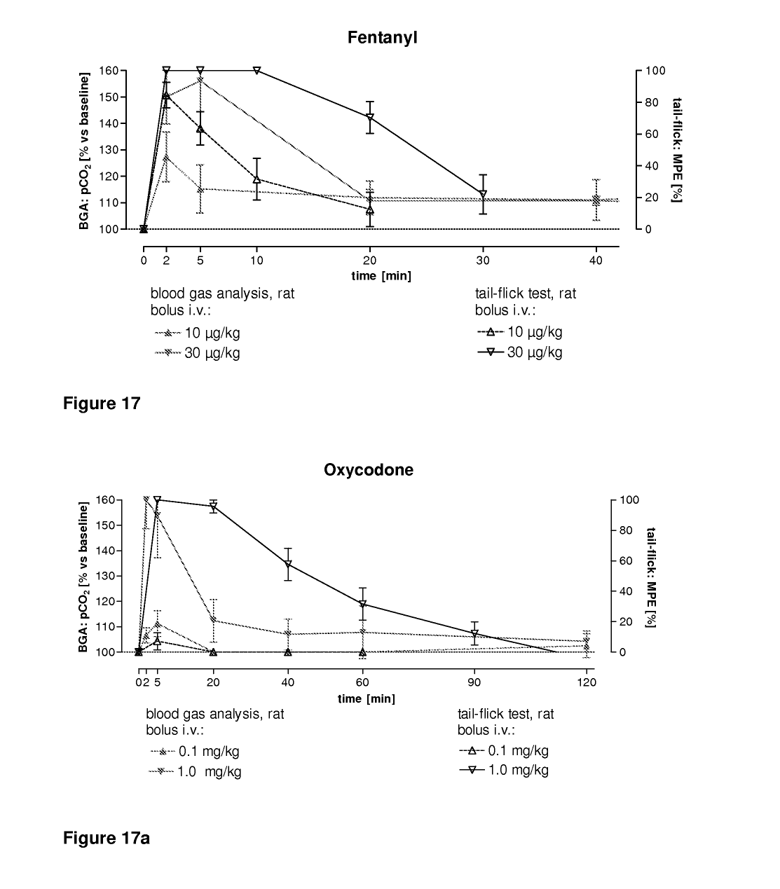

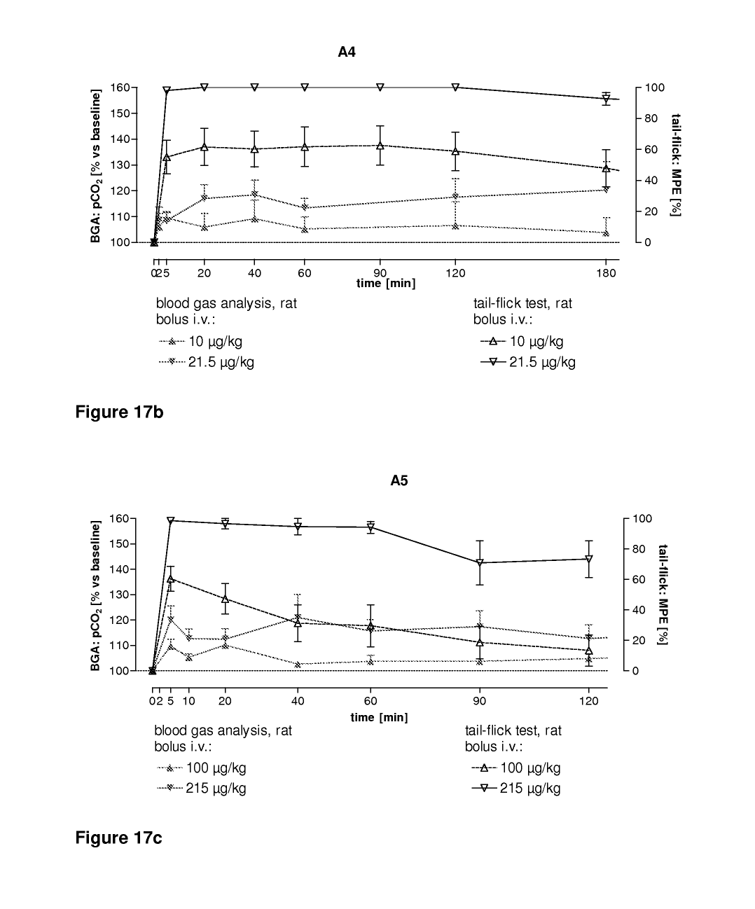

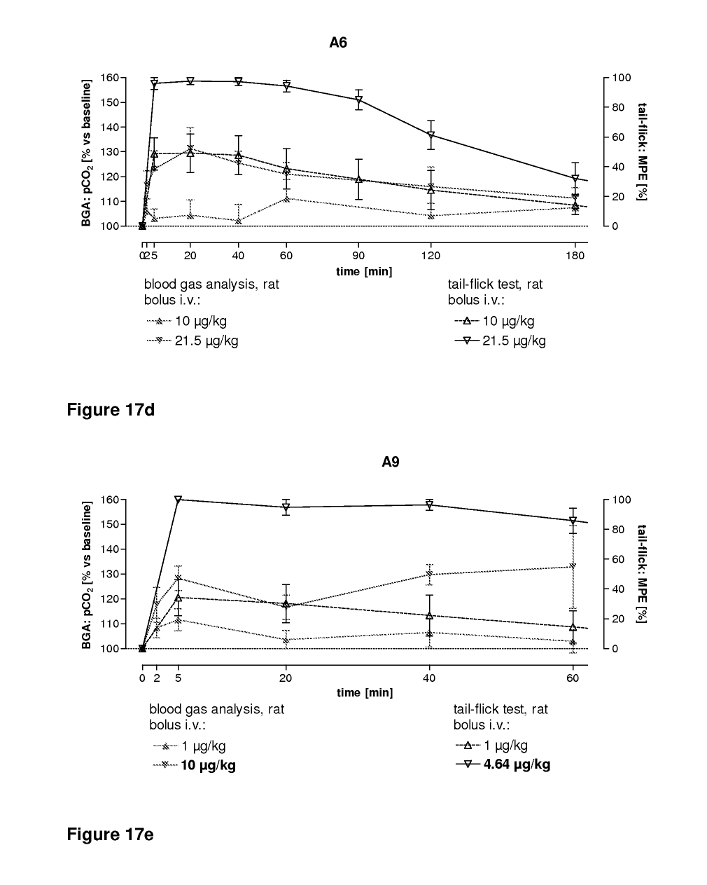

In the case of pure μ-opioids, exemplified by B1 (fentanyl, FIG. 17) and B4 (oxycodone, FIG. 17a), a distinct increase in arterial pCO₂ occurs at the time of maximum analgesic action due to μ-induced respiratory depression. At a 90-100% effective dose, the pCO₂ value rises by more than 50%.

In contrast, with mixed ORL1/μ-agonists such as A4, A5, A6 and A9, the pCO₂ value rises only slightly (FIGS. 17b-e). Even at a very high dosage, which is maximally analgesically active over several hours, arterial pCO₂ rises by only approx. 20-30%.

It has been shown by antagonization tests that

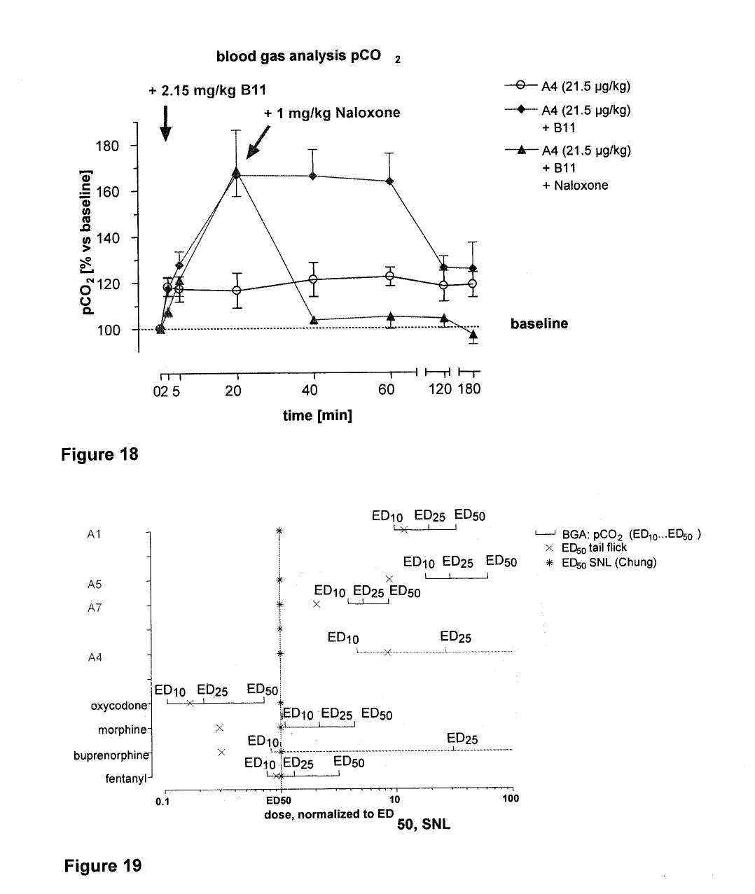

(1) respiratory depression is distinctly increased (approx. 70%) after antagonization of the ORL1 component, for example of A4 with B11, and
(2) respiratory depression is completely suppressed by subsequent μ-antagonization with naloxone (FIG. 18).

This shows that the reduced respiratory depression with mixed ORL1/μ-agonists with the characteristics according to the invention is attributable to the ORL1 component. Respiratory depression is entirely triggered by the μ component. The antagonization experiments demonstrate that the reduction in respiratory depression is effected by the ORL1 component.

Since, especially in anaesthesia, the respiratory depression triggered by μ-opioids may give rise to serious complications, it is preferred to use the compounds with the characteristics according to the invention for anaesthesia or concomitantly with anaesthesia. It is particularly preferred in this connection if the half-life of the compound is less than one hour, very particularly preferably less than 30 minutes.

The half-life is here taken to be the time in which half of the absorbed compound with the characteristics according to the invention has been metabolized and/or excreted.

There is also an increased risk of respiratory depression following surgery. By using the compounds with an ORL1/p ratio of 0.1 to 30, preferably of 0.1 to 20, at a Kᵢ value on the μ-opioid receptor of below 100 nM, higher dosages can be used postoperatively, and, if necessary, a more potent analgesia can thereby be achieved than with pure μ-agonists. It is therefore preferred to use the compounds with the characteristics according to the invention for the treatment of postoperative pain.

Since the risk of respiratory depression is distinctly increased in people aged 60 and above in comparison with younger people, as has been demonstrated by studies (Cepeda et al., Clinical Pharmacology & Therapeutics 2003, 74, 102-112), the compounds with an ORL1/μ-ratio of 0.1 to 30, preferably of 0.1 to 20, at a Kᵢ value on the μ-opioid receptor of below 100 nM are preferably used for the treatment of pain in patients over 60 years of age. It is thus particularly preferred to use the compounds with the characteristics according to the invention for anaesthesia, concomitantly with anaesthesia or postoperatively in patients over 60 years of age. The compounds are particularly preferably also used for the treatment of neuropathic pain in patients over 60 years of age.

The reduction in respiratory depression due to the ORL1 component may be demonstrated, as shown in the Examples, by antagonization experiments. The invention thus also provides the use of compounds which exhibit an affinity of at least 100 nM for the μ-opioid receptor and for the ORL1 receptor and, due to the ORL1 component, exhibit respiratory depression which is reduced in comparison with a μ-opioid of the same affinity range, for the treatment of pain, preferably concomitantly with anaesthesia or postoperatively.

Greater Safety Margins with Mixed ORL1/μ-Agonists

Thanks to the reduced μ-OR-mediated respiratory depression, on the one hand, and the increased efficacy against neuropathic pain, on the other hand, the mixed ORL1/μ-agonists are distinguished by distinctly enlarged safety margins relative to pure μ-opioids. For mixed ORL1/μ-agonists with the characteristics according to the invention, exemplified by Examples A1, A5, A7, A6 and A4, the threshold dose (ED₁₀) for an increase in arterial pCO₂ is higher by a factor of approx. 3 to 20 than the semimaximal active dose (ED₅₀) against neuropathic pain (FIG. 19). This means that, in particular in chronic pain states, due to the elevated efficacy of the compounds with the characteristics according to the invention, on the one hand, and the antiopioid component, on the other hand, the safety margin from the possible opioid side-effects is so large that μ-typical side-effects occur comparatively less frequently at identical efficacy in the therapeutic range.

Thanks to the high safety margin from the opioid side effects, the compounds are especially suitable for the treatment of pain in palliative patients. Palliative patients are especially affected by opioid side effects due to their multimorbid condition. The invention therefore also provides the use of compounds with an ORL1/μ ratio of 0.1 to 30, preferably of 0.1 to 20, at a Kᵢ value on the μ-opioid receptor of below 100 nM for the treatment of pain in palliative patients.

Compounds which exhibit an affinity for the μ-opioid receptor of at least 100 nM (Kᵢ value, human) and an affinity for the ORL-1 receptor, wherein the ratio between the affinities ORL1:μ (Kᵢ values) is between 1:10 and 30:1, preferably from 1:10 to 20:1, therefore in summary in particular exhibit the following advantages relative to standard therapy with μ-opioids:
enhancement of action against chronic pain, in particular, against neuropathic pain and against inflammatory pain,
distinctly reduced side-effects, for example, respiratory depression, withdrawal/addiction and opioid-induced hyperalgesia, at comparable efficacy against acute pain.

The compounds which exhibit an affinity for the μ-opioid receptor of at least 100 nM (K, value, human) and an affinity for the ORL-1 receptor, wherein the ratio between the affinities for ORL1:μ (Kᵢ values) is between 1:10 and 30:1, preferably from 1:10 to 20:1, have the above-stated characteristics. The observed advantages are not based on characteristics which are specifically possessed by the investigated compounds, these effects instead arising from the mode of action. It has proved possible to prove this by antagonization experiments, in which it has been shown that the ORL1 component makes a contribution to analgesia, but suppresses μ-typical side-effects. In the analgesic range, the ORL1 component acts synergistically, but in the range of the investigated side-effects in opposing manner. The decisive factor here is the ratio of the two components.

The values which define the range according to the invention relate to in vitro data; in those cases in which one or more active metabolites are formed in vivo, the metabolites may influence activity. If metabolites are formed, the following cases may be distinguished:

### a) Use of Prodrugs

Compounds which do not exhibit the binding profile according to the invention may form metabolites which exhibit an affinity for the μ-opioid receptor of at least 100 nM (Kᵢ value, human) and an affinity for the ORL-1 receptor, wherein the ratio between the affinities ORL1/μ defined as 1/[K_{i(ORL1)}/K_{i(μ)}] is between 1:10 and 30:1, preferably from 1:10 to 20:1, and therefore still exhibit the characteristics according to the invention. This may be established by determining the Kᵢ values of the metabolites. The invention accordingly also provides the use of compounds which form metabolites which exhibit an affinity for the μ-opioid receptor of at least 100 nM (Kᵢ value, human) and an affinity for the ORL-1 receptor, wherein the ratio between the affinities ORL1/μ defined as 1/[K_{i(ORL1)}/K_{i(μ)}] is between 1:10 and 30:1, preferably from 1:10 to 20:1, wherein the contribution to efficacy and/or to reducing μ-typical side-effects is detectable by antagonization experiments.

b) Formation of Metabolites which Jointly or Together with the Parent Substance give Rise to the Profile According to the Invention

If, for example, a selective μ-agonist is partially metabolized to yield a selective ORL1 agonist and if the resultant mixture exhibits the characteristics according to the invention, i.e. the ratio of ORL1/μ defined as 1/[K_{i(ORL1)}/K_{i(μ)}] is between 0.1 and 30 and the Kᵢ value on the human μ-opioid receptor is at least 100 nM, the mixture is likewise provided by the invention. These mixtures may also arise from compounds which exhibit no selectivity, but nevertheless lie outside the range according to the invention. The characteristics according to the invention may, on the one hand, be proven by determining the binding constants of the mixture which arises in vivo, wherein the concentrations may be determined by HPLC-MS investigations, and, on the other hand, by demonstrating the contribution made by the ORL1 component to the enhancement of action against chronic pain and/or to reducing μ-typical side-effects by antagonization experiments with an ORL1 antagonist. The compounds furthermore have the characteristic of being active against acute pain. The invention thus also provides mixtures of substances formed by metabolism which exhibit the characteristics according to the invention, wherein the binding constants of the mixture correspond to the range according to the invention and the contribution made to efficacy and/or to reducing μ-typical side-effects is detectable by antagonization experiments.

The actions effected by the compounds according to the invention may also be achieved by administration of two or more different substances. This may, on the one hand, be demonstrated by determining the binding constants of the mixture, and, on the other hand, by showing the contribution made by the ORL1 component to the enhancement of action against chronic pain and/or to reducing μ-typical side-effects by antagonization experiments with an ORL1 antagonist. The compounds furthermore have the characteristic of being active against acute pain. The invention accordingly also provides the use of a μ-agonist which is more selective than ORL1/μ defined as 1/[K_{i(ORL1)}/K_{i(μ)}] 0.1, and an ORL1 agonist which is more selective than ORL1/μ defined as 1/[K_{i(ORL1)}/K_{i(μ)}] 30, for the production of a medicament for the treatment of pain, wherein the combination has the characteristics of the compounds according to the invention, i.e. the combination or the combination of the metabolites thereof formed in vivo exhibits an affinity for the μ-opioid receptor of at least 100 nM (Kᵢ value, human) and an affinity for the ORL-1 receptor, wherein the ratio between the affinities ORL1/μ defined as 1/[K_{i(ORL1)}/K_{i(μ)}] is between 0.1 and 30, preferably from 0.1 to 20. Such a combination is preferably used for the treatment of neuropathic pain, in particular for the treatment of pain with postzoster neuralgia and diabetic polyneuropathy pain. Use of such a combination is furthermore preferred in anaesthesia. The stated combinations are particularly preferably used in people over 60 years of age.

Apart from at least one compound with the characteristics according to the invention or a combination according to the invention, the medicaments according to the invention optionally contain suitable additives and/or auxiliary substances, such as matrix materials, fillers, solvents, diluents, dyes and/or binders and may be administered as liquid dosage forms in the form of solutions for injection, drops or succi, as semisolid dosage forms in the form of granules, tablets, pellets, patches, capsules, dressings or aerosols. Selection of the auxiliary substances etc. and the quantities thereof which are to be used depends upon whether the medicament is to be administered orally, perorally, parenterally, intravenously, intraperitoneally, intradermally, intramuscularly, intranasally, buccally, rectally or topically, for example onto the skin, mucous membranes or into the eyes. Preparations in the form of tablets, coated tablets, capsules, granules, drops, succi and syrups are suitable for oral administration, while solutions, suspensions, easily reconstitutible dried preparations and sprays are suitable for parenteral, topical and inhalatory administration. Compounds according to the invention in a depot in dissolved form or in a dressing, optionally with the addition of skin penetration promoters, are suitable percutaneous administration preparations. Orally or percutaneously administrable preparations may release the compounds with the characteristics according to the invention or a combination according to the invention in delayed manner. In principle, other additional active ingredients known to the person skilled in the art may be added to the medicaments according to the invention.

The quantity of active substance to be administered to the patient varies as a function of patient weight, mode of administration, the indication and the severity of the condition. Conventionally, 0.005 to 20 mg/kg, preferably 0.05 to 5 mg/kg of at least one compound or combination with the characteristics according to the invention are administered.

Compounds A1 to A10, which all exhibit the characteristics according to the invention, fall within the group of spirocyclic cyclohexane derivatives. These compounds have an affinity for the μ-opioid receptor and/or for the ORL-1 receptor, but a subgroup of these compounds exhibits the characteristics according to the invention.

The invention therefore also provides a compound from the group of spirocyclic cyclohexane derivatives of the general formula I

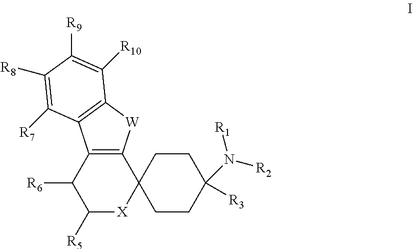

in which
R¹ and R² mutually independently denote H or CH₃, wherein R¹ and R² do not simultaneously denote H;
R³ denotes phenyl, benzyl or heteroaryl, in each case unsubstituted or monosubstituted or polysubstituted with F, Cl, OH, CN and/or OCH₃;
W denotes NR⁴, O or S;
and
   R⁴ denotes H; C₁₋₅ alkyl; phenyl; phenyl-C₁₋₃-alkyl; R¹²OC—C₁₋₃-alkyl, SO₂R¹²,
   wherein R¹² denotes H; C₁₋₇ aliphatic hydrocarbyl, which is branched or unbranched, saturated or unsaturated, and unsubstituted or monosubstituted or polysubstituted with OH, F and/or COOC₁₋₄ alkyl; C₄₋₆ cycloalkyl; aryl or heteroaryl, which is unsubstituted or monosubstituted or polysubstituted with F, Cl, Br, CF₃, OCH₃ and/or C₁₋₄ alkyl, which alkyl is branched or unbranched, and unsubstituted or monosubstituted or polysubstituted with F, Cl, CN, CF₃, N(CH₃)₂ and/or OH; or phenyl or heteroaryl, which is unsubstituted or monosubstituted or polysubstituted with F, Cl, Br, CF₃, OCH₃ and/or C₁₋₄ alkyl, which alkyl is branched or unbranched, wherein the phenyl or heteroaryl is attached via saturated or unsaturated C₁₋₃ aliphatic hydrocarbyl; or C₅₋₆ cycloalkyl attached via saturated or unsaturated C₁₋₃ aliphatic hydrocarbyl; OR¹³; or NR¹⁴R¹⁵;
R⁵ denotes H; COOR¹³, CONR¹³, OR¹³; C₁₋₅ aliphatic hydrocarbyl, which is saturated or unsaturated, branched or unbranched, and unsubstituted or monosubstituted or polysubstituted with OH, F, CF₃ and/or CN;
R⁶ denotes H;
or R⁵ and R⁶ together denote (CH₂)ₙ with n=2, 3, 4, 5 or 6, wherein individual hydrogen atoms may be replaced by F, Cl, NO₂, CF₃, OR¹³, CN and/or C₁₋₅ alkyl;
R⁷, R⁸, R⁹ and R¹⁰ mutually independently denote H, F, Cl, Br, NO₂, CF₃, OH, OCH₃, CN, COOR¹⁹, NR¹⁴R¹⁵; or C₁₋₅ alkyl; or heteroaryl, which is unsubstituted or monosubstituted or polysubstituted with benzyl, CH₃, Cl, F, OCH₃ and/or OH;
   wherein R¹³ denotes H or C₁₋₅ alkyl;
   R¹⁴ and R¹⁵ mutually independently denote H or C₁₋₅ alkyl;
X denotes O, S, SO, SO₂ or NR¹⁷;
   R¹⁷ denotes H; C₁₋₅ aliphatic hydrocarbyl, which is saturated or unsaturated, and branched or unbranched; COR¹² or SO₂R¹²,
      wherein the compound is optionally in the form of a pure diastereomer thereof, a racemate thereof, a pure enantiomer thereof, or in the form of a mixture of the stereoisomers thereof in any desired mixing ratio;
      and/or
      the compound is in the form of a base or salt thereof, in particular the physiologically acceptable salts, or salts of physiologically acceptable acids or cations;
      which exhibits an affinity for the μ-opioid receptor of at least 100 nM (Kᵢ value, human) and an affinity for the ORL-1 receptor, wherein the ratio between the affinities ORL1/μ defined as 1/[K_{i(ORL1)}/K_{i(μ)}] is from 0.1 to 30, for the treatment of diabetic polyneuropathy pain, postoperative pain or pain with postzoster neuralgia.

The invention will now be described in greater detail with the following non-limiting examples, which are provided for illustrative purposes only.

### EXAMPLES

### Abbreviations Used

### AUC Area Under Curve

### CFA Complete Freund's Adjuvant

DBTC Dibutyltin dichloride

### MPE Maximum Possible Effect

The following Examples illustrate the invention. Typical representatives of μ-agonists, mixed μ/ORL1 agonists, ORL1 agonists and an ORL1 antagonist were used. The μ-antagonist used was the clinically used compound naloxone. These exemplary compounds were subjected to numerous investigations which demonstrate the exceptional position of the compounds with the characteristics according to the invention.

| | | Source or production |
|---|---|---|
| Name | Structure | process |
| B1 (fentanyl) | 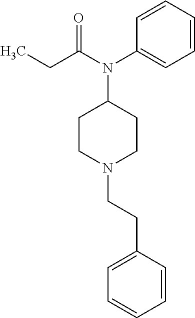 | Commercially obtainable |
| B2 (sufentanil) | 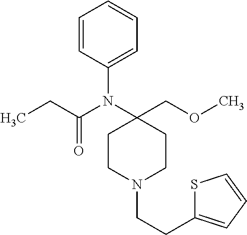 | Commercially obtainable |
| B3 (morphine) | 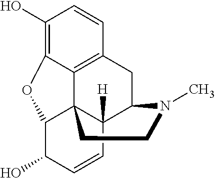 | Commercially obtainable |
| B4 (oxycodone) | 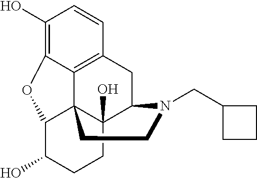 | Commercially obtainable |
| B5 (buprenorphine) | 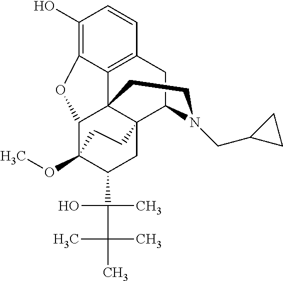 | Commercially obtainable |
| B6 (hydromorphone) | 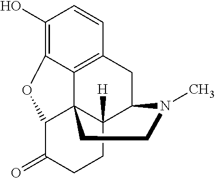 | Commercially obtainable |
| B7 (L-methadone) | 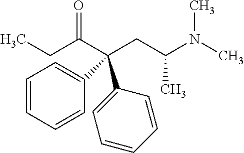 | Commercially obtainable |
| B8 | 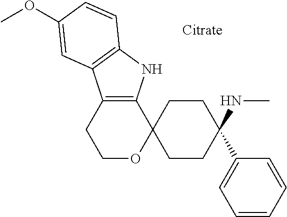 | Synthesis similar to A1 |
| A1 | 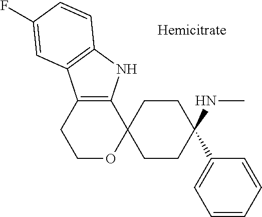 | Example 49, EP1560835 1,1-(3-methylamino- 3-phenylpenta- methylene)-6-fluoro- 1,3,4,9-tetrahydro- pyrano[3,4-b]indole hemicitrate |
| A2 | 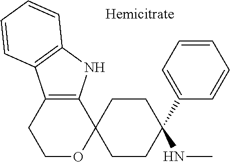 | Example 28, EP1560835 1,1-(3-methylamino-3- phenylpenta-methylene)- 1,3,4,9-tetrahydro- pyrano[3,4-b]indole hemicitrate |
| A3 | 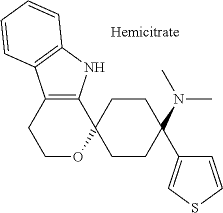 | Example 8, WO200566183 1,1-[3-dimethylamino-3-(3- thienyl)penta-methylene]- 1,3,4,9-tetrahydro- pyrano[3,4-b]indole hemicitrate |
| A4 | 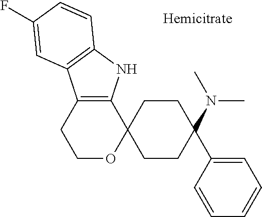 | Example 24, EP1560835 1,1-(3-dimethylamino- 3-phenylpenta- methylene)-6-fluoro- 1,3,4,9-tetrahydro- pyrano[3,4-b]indole hemicitrate, |
| A5 | 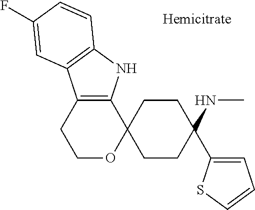 | Example 15, WO200566183 1,1-[3-methylamino-3- (2-thienyl)penta- methylene]-1,3,4,9- tetrahydro- pyrano[3,4-b]-6- fluoroindole citrate |
| A6 | 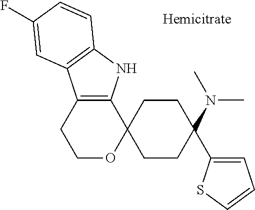 | Example 10, WO200566183 1,1-[3-dimethyl- amino-3-(2- thienyl)penta- methylene]-1,3,4,9- tetrahydro- pyrano[3,4-b]-6- fluoroindole hemicitrate |
| A7 | 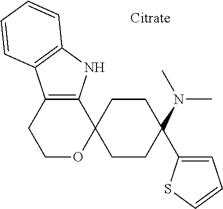 | Example 7, WO200566183 1,1-[3-dimethylamino- 3-(2-thienyl)penta- methylene]-1,3,4,9- tetrahydropyrano[3,4- b]indole citrate |
| A8 | 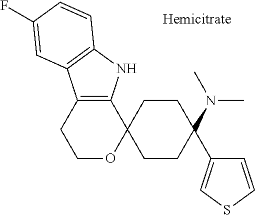 | Example 13, WO200566183 1,1-[3-dimethyl-amino-3-(3- thienyl)penta-methylene]- 1,3,4,9-tetrahydro- pyrano[3,4-b]-6- fluoroindole hemicitrate |
| A9 | 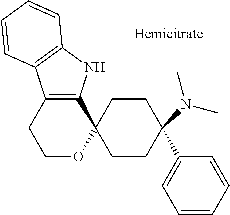 | Example 3, EP1560835 1,1-(3-dimethyl-amino-3- phenylpenta-methylene)- 1,3,4,9-tetrahydro- pyrano[3,4-b]indole hemicitrate |
| A10 | 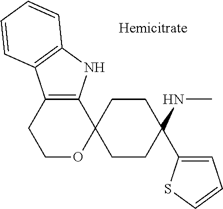 | Example 14, WO200566183 1,1-[3-methylamino- 3-(2-thienyl)penta- methylene]-1,3,4,9- tetrahydro- pyrano[3,4-b]indole citrate |
| A11 | 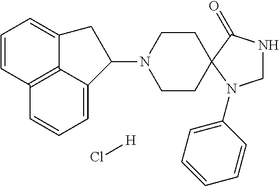 | EP 08856514 |
| B9 | 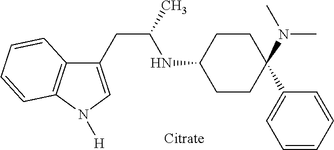 | Example 59, EP1392641 + separation of enantiomers |
| B10 (nociceptin) | Peptide, endogenous | Commercially |
| | ligand | obtainable |
| B11 | 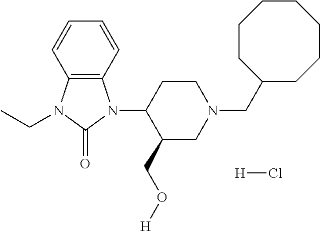 | ORL1 antagonist WO9854168 |

### Measurement of ORL1 Binding

The cyclohexane derivatives of the general formula I were investigated in a receptor binding assay with ³H-nociceptin/orphanin FQ with membranes from recombinant CHO-ORL1 cells. This test system was carried out in accordance with the method presented by Ardati et al. (Mol. Pharmacol., 51, 1997, pp. 816-824). The concentration of ³H-nociceptin/orphanin FQ in these tests was 0.5 nM. The binding assays were in each case performed with 20 μg of membrane protein per 200 μl batch in 50 mM Hepes, pH 7.4, 10 mM MgCl₂ and 1 mM EDTA. Binding to the ORL1 receptor was determined using 1 mg portions of WGA-SPA Beads (Amersham-Pharmacia, Freiburg), by one hour's incubation of the batch at room temperature and subsequent measurement in a Trilux scintillation counter (Wallac, Finland). The affinity is stated in Table 1 as the nanomolar Kᵢ value or % inhibition at c=1 μM.

### Measurement of μ Binding

Receptor affinity for the human μ-opiate receptor was determined in a homogeneous batch in microtiter plates. To this end, dilution series of the particular substance to be tested were incubated at room temperature for 90 minutes in a total volume of 250 μl with a receptor membrane preparation (15-40 μg of protein per 250 μl of incubation batch) of CHO-K₁ cells, which express the human μ-opiate receptor (RB-HOM receptor membrane preparation from NEN, Zaventem, Belgium) in the presence of 1 nmol/l of the radioactive ligand [³H]-naloxone (NET719, from NEN, Zaventem, Belgium) and of 1 mg of WGA-SPA beads (wheat germ agglutinin SPA beads from Amersham/Pharmacia, Freiburg, Germany). The incubation buffer used was 50 mmol/l tris-HCl supplemented with 0.05 wt. % of sodium azide and with 0.06 wt. % of bovine serum albumin. 25 μmol/l of naloxone were additionally added to determine nonspecific binding. Once the ninety minute incubation time had elapsed, the microtiter plates were centrifuged off for 20 minutes at 1000 g and the radioactivity measured in a β-Counter (Microbeta-Trilux, from PerkinElmer Wallac, Freiburg, Germany). The percentage displacement of the radioactive ligand from its binding to the human μ-opiate receptor was determined at a concentration of the substances to be tested of 1 μmol/l and stated as percentage inhibition (% inhibition) of specific binding. In some cases, on the basis of the percentage displacement by different concentrations of the compounds to be tested of the general formula I, IC₅₀ inhibition concentrations which bring about 50% displacement of the radioactive ligand were calculated. Kᵢ values for the test substances were obtained by conversion using the Cheng-Prusoff equation.

The Kᵢ values of the Example compounds are summarized in the following Table.

Comparison of analgesic efficacy (as ED₅₀, % MPE) in the acute
pain model (tail flick, rat/mouse) and in neuropathic pain
models (Chung, rat; Bennett, rat/mouse):

| Substance | Kᵢ (ORL1) | | Kᵢ (μ-OR) | | Ratio ORL1:μ¹ ≈ | Classification |
|---|---|---|---|---|---|---|
| B1 (fentanyl) | 1600 | nM | 7.9 | nM | — | μ-agonist |
| B2 (sufentanil) | 145 | nM | 0.8 | nM | — | μ-agonist |
| B3 (morphine) | >1 | μM | 9 | nM | — | μ-agonist |
| B4 (oxycodone) | >10 | μM | 130 | nM | — | μ-agonist |
| B5 (buprenorphine) | 36 | nM | 0.3 | nM | — | opioid agonist (weak |
| | | | | | | ORL1 component) |
| B6 (hydromorphone) | >10 | μM | 4 | nM | — | μ-agonist |
| B7 (L-methadone) | >1 | μM | 7 | nM | — | μ-agonist |
| B8 | 70 | nM | 2.4 | nM | 0.03 | μ-agonist, weak ORL1 |
| | | | | | (1:30) | agonist |
| A1 | 14 | nM | 1.8 | nM | 0.1 | mix. ORL1/μ-agonist |
| | | | | | (1:10) | |
| A2 | 1.7 | nM | 0.4 | nM | 0.25 | mix. ORL1/μ-agonist |
| | | | | | (1:4) | |
| A3 | 0.3 | nM | 0.1 | nM | 0.3 | mix. ORL1/μ-agonist |
| | | | | | (1:3) | |
| A4 | 2 | nM | 1 | nM | 0.5 | mix. ORL1/μ-agonist |
| | | | | | (1:2) | |
| A5 | 2 | nM | 1 | nM | 0.5 | mix. ORL1/μ-agonist |
| | | | | | (1:2) | |
| A6 | 1 | nM | 1 | nM | 1 | mix. ORL1/μ-agonist |
| | | | | | (1:1) | |
| A7 | 0.4 | nM | 0.3 | nM | 1 | mix. ORL1/μ-agonist |
| | | | | | (1:1) | |
| A8 | 0.5 | | 1.3 | nM | 2 | mix. ORL1/μ-agonist |
| | | | | | (2:1) | |
| A9 | 0.5 | nM | 1 | nM | 2 | mix. ORL1/μ-agonist |
| | | | | | (2:1) | |
| A10 | 0.2 | nM | 0.5 | nM | 2 | mix. ORL1/μ-agonist |
| | | | | | (2:1) | |
| A11 | 1 | nM | 23 | nM | 20 | ORL1 agonist; |
| | | | | | (20:1) | comparatively weak μ |
| | | | | | | component |
| B9 | 0.4 | nM | 55 | nM | 140 | ORL1 agonist; |
| | | | | | (140:1) | comparatively weak μ |
| | | | | | | component |
| B10 (nociceptin) | 0.3 | nM | ~250 | nM | 800:1 | ORL1 agonist; |
| | | | | | | endogenous ligand |

¹Definition: 1/[K_{i(ORL1)}/K_{i(μ)}]

### Analgesic Testing by Tail Flick Test in Mice

The analgesic efficacy of the test compound was investigated in the thermal radiation (tail flick) test in mice in accordance with the method of D'Amour and Smith (J. Pharm. Exp. Ther. 72, 74-79 (1941)). NMRI mice weighing between 20 and 24 g were used for this purpose. The mice were individually put in special test cages and the base of the tail was exposed to the focused thermal radiation from an electric lamp (tail flick type 55/12/10.fl, Labtec, Dr. Hess). The lamp intensity was adjusted such that the time from switching on of the lamp until sudden flicking away of the tail (pain latency) in untreated mice amounted to 2.5 to 5 seconds. Before administration of a test compound, the animals were pretested twice within 30 minutes and the mean of these measurements was calculated as a pretest mean. Pain measurement was carried out 20, 40 and 60 min after intravenous administration. Analgesic action was determined as the increase in pain latency (% MPE) in accordance with the following formula:

[(T₁−T₀)/(T₂−T₀)]×100

T₀ is here the latency time before and T₁ the latency time after administration of the substance, T₂ is the maximum exposure time (12 sec).

In order to determine dose dependency, the test compound was administered in 3-5 logarithmically increasing doses, which in each case included the threshold and the maximum active dose, and the ED₅₀ values were determined using regression analysis. ED₅₀ was calculated at maximum action 20 minutes after intravenous substance administration.

### Analgesic Testing by Tail Flick Test in Rats

The analgesic efficacy of the test compounds was investigated in the thermal radiation (tail flick) test in rats in accordance with the method of D'Amour and Smith (J. Pharm. Exp. Ther. 72, 74-79 (1941)). Sprague-Dawley females weighing between 134 and 189 g were used for this purpose. The animals were individually put in special test cages and the base of the tail was exposed to the focused thermal radiation from a lamp (tail flick type 50/08/1.bc, Labtec, Dr. Hess). The lamp intensity was adjusted such that the time from switching on of the lamp until sudden flicking away of the tail (pain latency) in untreated mice amounted to 2.5 to 5 seconds. Before administration of a test compound, the animals were pretested twice within 30 minutes and the mean of these measurements was calculated as a pretest mean. Pain measurement was carried out 20, 40 and 60 min after intravenous administration. Analgesic action was determined as the increase in pain latency (% MPE) in accordance with the following formula:

[(T₁−T₀)/(T₂−T₀)]×100

T₀ is here the latency time before and T₁ the latency time after administration of the substance, T₂ is the maximum exposure time (12 sec).

In order to determine dose dependency, the particular test compound was administered in 3-5 logarithmically increasing doses, which in each case included the threshold and the maximum active dose, and the ED₅₀ values were determined using regression analysis. ED₅₀ was calculated at maximum action, 20 minutes after intravenous substance administration.

Tail Flick Test with Reduced Intensity of Thermal Radiation in Rats

The modulatory efficacy of the test compounds in response to acute, noxious thermal stimuli was investigated in the thermal radiation (tail flick) test in rats in accordance with the method of D'Amour and Smith (J. Pharm. Exp. Ther. 72, 74-79 (1941)). Male Sprague-Dawley rats (breeder: Janvier, Le Genest St. Isle, France) weighing between 200 and 250 g were used for this purpose. The animals were individually accommodated in special test compartments and the base of the tail was exposed to focused thermal radiation from an analgesia meter (model 2011, Rhema Labortechnik, Hofheim, Germany). The size of the group was 10 animals. The intensity of thermal radiation was adjusted such that the time from switching on the thermal radiation until sudden withdrawal of the tail (withdrawal latency) in untreated animals was approx. 12-13 seconds. Before administration of a substance according to the invention, withdrawal latency was determined twice at an interval of five minutes and the mean defined as the control latency time. Tail withdrawal latency was measured for the first time 10 minutes after intravenous substance administration. Once the antinociceptive effect had subsided (after 2-4 hours), the measurements were performed at 30 minute intervals up to at most 6.5 hours after administration of the substance. Antinociceptive or pronociceptive action was determined respectively as an increase or decrease in withdrawal latency in accordance with the following formula:

(% MPE)=[(T₁−T₀)/(T₂−T₀)]×100

Definitions: T₀: control latency time before administration of the substance, T₁: latency time after administration of the substance, T₂: maximum exposure time to the thermal radiation (30 seconds), MPE: maximum possible effect.

Statistically significant differences between the substance and vehicle group were tested by analysis of variance (repeated measures ANOVA). The significance level was set at ≦0.05.

Chung model: Mononeuropathic Pain after Spinal Nerve Ligature

Animals: Male Sprague-Dawley rats (140-160 g) from a commercial breeder (Janvier, Genest St. Isle, France), were kept under a 12:12 h light:dark cycle. The animals were provided with feed and tap water ad libitum. An interval of one week was left between delivery of the animals and surgery. After surgery, the animals were tested repeatedly for a period of 4-5 weeks, a wash-out time of at least one week being observed.

Description of model: Under pentobarbital anaesthesia (Narcoren®, 60 mg/kg i.p., Merial GmbH, Hallbergmoos, Germany), the left L5, L6 spinal nerves were exposed by removing a piece of the paravertebral muscle and some of the left side spinal process of the L5 lumbar vertebra. The L5 and L6 spinal nerves were carefully isolated and tied off with a strong ligature (NC-silk black, USP 5/0, metric 1, Braun Melsungen AG, Melsungen, Germany) (Kim and Chung 1992). After application of the ligature, the muscle and adjacent tissue were stitched up and the wound closed by means of metal clips. After one week's convalescence, the animals placed in cages with a wire floor to measure mechanical allodynia. The withdrawal threshold was determined on the ipsilateral and/or contralateral hind paw using an electronic von Frey filament (Somedic A B, Malmö, Sweden). The median of five stimulations constituted a measurement time. The animals were tested 30 min before and at different times after administration of the test substance or vehicle solution. The data were determined as a percentage of the maximum possible effect (% MPE) from pretesting of individual animals (=0% MPE) and the test values for an independent sham control group (=100% MPE). Alternatively, the withdrawal thresholds were stated in grams.

Statistical evaluation: ED₅₀ values and 95% confidence intervals were determined by semilogarithmic regression analysis at the time of maximum effect. The data were subjected to analysis of variance with repeated measures and post hoc Bonferroni analysis. The size of the group was usually n=10.

References: Kim, S. H. and Chung, J. M., An experimental model for peripheral neuropathy produced by segmental spinal nerve ligation in the rat, Pain, 50 (1992) 355-363.

### Bennett Model: Neuropathic Pain in Mice or in Rats

Efficacy against neuropathic pain was investigated in the Bennett model (chronic constriction injury; Bennett and Xie, 1988, Pain 33: 87-107).

Sprague-Dawley rats weighing 140-160 g are provided under Narcoren anaesthesia with four loose ligatures of the right ischial nerve. NMRI mice weighing 16-18 g are provided under Ketavet-Rompun anaesthesia with three loose ligatures of the right ischial nerve. On the paw innervated by the damaged nerve, the animals develop hypersensitivity which, after one week's convalescence, is quantified over a period of approx. four weeks by means of a cold metal plate at 4° C. (cold allodynia). The animals are observed on this plate for a period of 2 min. and the number of withdrawal responses by the damaged paw is measured. Relative to the preliminary value prior to administration of the substance, the action of the substance is determined on four occasions over a period of one hour (for example 15, 30, 45, 60 min. after administration) and the resultant area under the curve (AUC) and the inhibition of cold allodynia at the individual measuring points is stated as a percentage action relative to the vehicle control (AUC) or to the initial value (individual measurement points). The size of the group is n=10, the significance of an antiallodynic action (*=p<0.05) is determined with reference to an analysis of variance with repeated measures and post hoc Bonferroni analysis.

### Vincristine-Induced Polyneuropathy

The model is described in the literature (T. Christoph, B. Koegel, K. Schiene, M. Meen, J. De Vry, E. Friderichs, European Journal of Pharmacology, 2005, 507, 87-98).

### Diabetic Polyneuropathy Pain

The model is described in the literature (T. Christoph, B. Koegel, K. Schiene, M. Meen, J. De Vry, E. Friderichs, European Journal of Pharmacology, 2005, 507, 87-98).

Relative enhancement of action in models of neuropathic pain

| | | | | | Enhance- |
|---|---|---|---|---|---|
| | | | | Route of | ment |
| | Ratio | | ED₅₀ | adminis- | of action |
| Substance | ORL1/μ | ED₅₀ acute | chronic | tration | factor |
| B3 | <1:100 | 1.1 | 3.7 | i.v. | 0.3× |
| (morphine) | | mg/kg¹ | mg/kg⁴ | | |
| B3 | <1:100 | 1.1 | 1.3 | i.v. | 0.8× |
| (morphine) | | mg/kg¹ | mg/kg⁵ | | |
| B3 | <1:100 | 2 | ~10 | i.th. | 0.5× |
| (morphine) | | μg/animal³ | μg/animal⁴ | | |
| B4 | <1:100 | 360 | 2170 | i.v. | 0.2× |
| (oxycodone) | | μg/kg¹ | μg/kg⁴ | | |
| B4 | <1:100 | 360 | 900 | i.v. | 0.4× |
| (oxycodone) | | μg/kg¹ | μg/kg⁵ | | |
| B4 | <1:100 | 670 | 2520 | i.p. | 0.3× |
| (oxycodone) | | μg/kg¹ | μg/kg⁴ | | |
| B4 | <1:100 | 670 | 1290 | i.p. | 0.5× |
| (oxycodone) | | μg/kg¹ | μg/kg⁵ | | |
| B6 (hydro- | <1:100 | 150 | 220 | i.v. | 0.7× |
| morphone) | | μg/kg¹ | μg/kg⁴ | | |
| B7 (L-meth- | <1:100 | 210 | 490 | i.v. | 0.4× |
| adone) | | μg/kg¹ | μg/kg⁴ | | |
| B5 (bupre- | <1:100 | 17 | 55 | i.v. | 0.3× |
| norphine) | | μg/kg¹ | μg/kg⁴ | | |
| B1 | <1:100 | 10 | 11 | i.v. | 0.9× |
| (fentanyl) | | μg/kg¹ | μg/kg⁴ | | |
| B1 | <1:100 | 43 | 230 | i.p. | 0.2× |
| (fentanyl) | | μg/kg¹ | μg/kg⁴ | | |
| B8 | 1:30 | 330 | 363 | i.v. | 0.9× |
| | | μg/kg¹ | μg/kg⁴ | | |
| Al | 1:10 | 110 | 9 | i.v. | 12× |
| | | μg/kg¹ | μg/kg⁴ | | |
| Al | 1:10 | 110 | 11 | i.v. | 10× |
| | | μg/kg¹ | μg/kg⁵ | | |
| A4 | 1:2 | 7 | 1 | i.v. | 7× |
| | | μg/kg¹ | μg/kg⁴ | | |
| A4 | 1:2 | 7 | 1 | i.v. | 7× |
| | | μg/kg¹ | μg/kg⁵ | | |
| A5 | 1:2 | 71 | 8 | i.v. | 8× |
| | | μg/kg¹ | μg/kg⁴ | | |
| A5 | 1:2 | 71 | 10 | i.v. | 7× |
| | | μg/kg¹ | μg/kg⁵ | | |
| A6 | 1:1 | 9 | 4 | i.v. | 2.5× |
| | | μg/kg¹ | μg/kg⁴ | | |
| A6 | 1:1 | 9 | 1 | i.v. | 9× |
| | | μg/kg¹ | μg/kg⁵ | | |
| A7 | 1:1 | 2 | 1 | i.v. | 2× |
| | | μg/kg^{l} | μg/kg⁴ | | |
| A9 | 2:1 | 2 | 0.8 | i.v. | 2.5× |
| | | μg/kg¹ | μg/kg⁴ | | |
| A11 | 20:1 | >10 | 0.2 | i.th. | >50× |
| | | μg/animal² | μg/animal⁶ | | |
| A11 | | 420 | no data | i.v. | |
| | | μg/kg² | | | |

¹tail flick model, rat
²tail flick model, mouse
³soft tail flick model, rat
⁴Chung model, rat
⁵Bennett model, rat
⁶Bennett model, mouse

For the purpose of graph plotting, the ED₅₀ value from the tail flick test and from the neuropathic pain models were normalized to the ED₅₀ value in the tail flick test in order to represent the relationship between the particular semimaximal active dosages (see FIGS. 1 and 2).

### Antagonization of the μ- and the ORL1 Component in the Chung Model

In antagonization experiments, partial antagonization with naloxone (μ-OR) and B11 (ORL1-R) was shown in each case. The data demonstrate that both components contribute to analgesia (see FIG. 3).

The analgesic efficacy of A4 remains in place even at a very high dosage of B11, i.e. with the ORL1 mode of action completely blocked.

FIG. 4 shows that, by antagonization of the μ- or ORL1 component respectively of A6, A5 and A1 with naloxone or B11, analgesic action of the non-antagonized component in each case remains in place.

### Separation of Antinociceptive and Antiallodynic Effect in Neuropathic Animals: Comparison of A4 and Morphine in Neuropathic Animals

In the Chung model, it is possible to differentiate between antinociceptive (contralateral) and antiallodynic (ipsilateral) action by comparative testing of the pain response on the ipsilateral and on the contralateral paw.

In the case of morphine, a purely antiallodynic action could be observed only once 1 mg/kg i.v. had been administered. Maximum efficacy here amounts to 29% MPE. Onset of a distinct antinociceptive action is already observed at the next highest test dose (2.15 mg/kg i.v.) (FIGS. 5, 5a).

In contrast, the maximum, purely antiallodynic action of A4 is 56% MPE. This is achieved at a test dose of 1 μg/kg i.v. (FIGS. 6, 6a).

Conclusion: A significantly stronger antiallodynic action is achieved due to the ORL1 component than with pure μ-opioids.

### Direct Comparison of A4 and Morphine in Naive and Neuropathic Animals

In order to exclude a possible influence of “pain quality” (tail flick, nociceptive stimulus vs. Chung, tactile allodynia) in the comparison of differing efficacy against acute pain and neuropathic pain, A4 and morphine were subjected to comparative testing in animals with a spinal nerve ligature (Chung model) and sham operated animals. In every case, the pain model used was the tail flick. The direct comparison shows that, once neuropathy has developed, the efficacy of morphine declines (which corresponds to the clinical situation), whereas it increases for A4 (see FIGS. 7 and 8).

While both substances exhibit comparable efficacy against acute pain (see below), the antiallodynic efficacy of A4 is higher than that of fentanyl by a factor of approx. 10.

### Comparison of Cytostatic-Induced Polyneuropathy Pain and Diabetes-Induced Polyneuropathy Pain

Against vincristine-induced polyneuropathy pain in the rat, A4 shows a significant efficacy at a dosage of 1 μg/kg (FIG. 23). At a dosage of 0.464 mg/kg no significant efficacy is yet to be observed (14.7±10.2% MPE). Against diabetes-induced neuropathy pain, on the other hand, a significant efficacy is already observed for the lowest dosage investigated (0.316 μg/kg) (FIG. 25). In this dose range no antinociceptive effect is yet to be observed. The comparison substances used clinically, morphine and pregabalin, show efficacy against diabetic polyneuropathy pain only in a dose range in which an antinociceptive effect is also to be observed (FIGS. 26, 27).

Enhancement of Action against Inflammatory Pain by Mixed ORL1/μ-Agonists
a) Single Motor Unit Discharges in Spinalized Rats. Comparison of Naive Animals and Animals after Carrageenan-Induced Inflammation.

The model is described in the literature (Herrero & Headley, 1996, Br J Pharmacol 118, 968-972).

24 h after induction of inflammation (100 μl carrageenan, 1%, intraplantar), the antinociceptive action of A4 (measured as inhibition of SMU activity after mechanical (pinch) or electrical (wind-up) stimulation) is distinctly increased (see FIGS. 10 and 10a). The antinociceptive action of morphine, in contrast, does not change after inflammation (see FIGS. 11 and 11a).

It furthermore also proved possible in this model to show an enhancement of action for A11 after induction of inflammation.

### CFA-Induced Hyperalgesia

### Complete Freund's Adjuvant (CFA) Induced Hyperalgesia in Rats

CFA-induced hyperalgesia is an animal model of chronic inflammatory pain. Male Sprague-Dawley rats (150-180 g) are given a single subplantar injection of 100 μl of thermally killed and dried mycobacteria (Mycobacterium tuberculosis; H37 Ra) in a mixture of paraffin oil and mannide monooleate as emulsifier (complete Freund's adjuvant, CFA) (dose 1 mg/ml). One day after the CFA injection, tactile hyperalgesia is verified with the assistance of an electronic von Frey hair (Somedic Sales AB, Hörby, Sweden). For this purpose, the animals are placed in a plastic box with a grating floor which allows free access to both hind paws. Subplantar stimulation is applied to the paw with the von Frey filament. In order to quantify the sensitivity of both the ipsilateral and the (untreated) contralateral paw to the mechanical stimulus, the paw withdrawal threshold is stated in grams of pressure applied. For each paw, stimulation is repeated 4× at an interval of 30 seconds in each case. The median of the four measured values is calculated. The withdrawal threshold of the ipsilateral and contralateral paw is determined at different times after CFA injection (1 h, 3 h, 1st day, 4th day), before (=preliminary value) and at different times after substance administration (measured value). The control is provided by a group of animals to which solvent is administered. The efficacy of a substance is calculated as % inhibition of hyperalgesia and furthermore as MPE % in the following manner:

% inhibition of HA=(1−HA measured value/HA preliminary value)×100

HA preliminary value=withdrawal threshold contralateral−withdrawal threshold ipsilateral before substance administration
HA measured value=withdrawal threshold contralateral−withdrawal threshold ipsilateral after substance administration

% MPE=[(WSs ipsi−WSo ipsi)/WSo contra−WSo ipsi]×100

WSo contra=withdrawal threshold of contralateral, untreated paw
WSo ipsi=withdrawal threshold of ipsilateral, untreated paw
WSs ipsi=withdrawal threshold of ipsilateral, treated paw after substance administration

MPE %: percent of the maximum possible effect; the maximum possible effect is defined as the withdrawal threshold of the contralateral, untreated paw.

In total, 10 rats are used per test group (substance and control). The mean±SEM is calculated from the medians of the individual animals. Significance is calculated by means of two-factor ANOVA for repeated measures. The significance of the interaction of substance-administration (treatment), time, time*treatment is analyzed with Wilks' lambda statistics. If a treatment effect is significant, a Fischer's test with a subsequent post hoc Dunnett's test is carried out.

While morphine tends to exhibit a slight decline in the antihyperalgesic action or a constant antinociceptive action over the investigation period, the antihyperalgesic and antinociceptive actions of A4 increase over 24 h. The effect is stable for at least 4 days (see FIGS. 12, 12a).

### Mustard Oil-Induced Visceral Inflammatory Pain in Mice

Male NMRI mice (body weight 20-35 g) are habituated for approx. thirty minutes on a grating in acrylic sheet cages (14.5×14.5 cm, height 10 cm). The behavior of the mice in response to ten instances of mechanical stimulation by means of von Frey filaments (1, 4, 8, 16, 32 mN) on the abdominal wall is recorded as a preliminary value. Behavior is analyzed either by means of the sum of the number of nocifensive responses or by means of the quality of these nocifensive responses and their weighting by multiplying the number of responses by the associated factor (factor 1: slight raising of abdomen, licking at site of stimulation, walking away; factor 2: stretching out hind paw, slight hopping away, twitching the hind paw, jerky, vigorous licking of the site of stimulation; factor 3: jumping away, vocalization) and subsequent summation.

Test substance or vehicle is then administered using a suitable mode of administration at a suitable time, depending on the substance's kinetics, before administration of the mustard oil. The size of the group is usually n=7.

Acute colitis is induced by rectal administration of 50 μl of mustard oil (3.5% in PEG200). Two to twelve minutes after administration of mustard oil, the animals exhibit spontaneous visceral pain behavior, which is observed. The number of responses is multiplied by the associated factor (factor 1: licking of abdominal wall; factor 2: stretching, pressing abdomen against the floor, bridge posture, contraction of the abdomen, backward movement or contraction of flank muscles) and then the sum is calculated, which represents the spontaneous visceral pain score. Instead of mustard oil, one group of animals receives a rectal administration of 50 μl of PEG200.

Twenty to forty minutes after administration of mustard oil, the behavior of the animals in response to ten instances of mechanical stimulation by means of von Frey filaments (1, 4, 8, 16, 32 mN) on the abdominal wall is observed and quantified as described above. Transferred mechanical allodynia is here determined from the sum of the responses on the stimulation with the 1 mN strength von Frey filament. Transferred mechanical hyperalgesia is determined as the sum of the weighted responses to stimulation with the 16 mN strength von Frey filament.

The action of the test substance in comparison with vehicle is described by 1. inhibition of spontaneous visceral pain behavior, 2. inhibition of transferred mechanical allodynia and 3. inhibition of transferred mechanical hyperalgesia.

The data are investigated by multifactorial analysis of variance with repeated measures and, if a significant action of the test substance (P<0.05) is found, the individual data are checked for significance by post hoc Bonferroni analysis. In the case of dose-response curves, ED₅₀ values, which describe the dose having semimaximal action, may be determined by linear regression analysis (after Christoph et al., 2005, Eur. J. Pharmacol. 507: 87-98).

Comparative testing of A4 and fentanyl in a model of transferred allodynia and transferred hyperalgesia in mice after non-neurogenic visceral inflammation induced by mustard oil revealed significantly higher efficacy of the mixed ORL1/μ-agonists for all three pain parameters, but especially for allodynia and hyperalgesia, in comparison with the pure μ-opioid.

Transferred allodynia

| | Ratio | | ED₅₀ visceral | Enhancement of |
|---|---|---|---|---|
| Substance | ORL1/μ | ED₅₀ acute | pain | action factor |
| B1 (fentanyl) | <1:100 | 30 μg/kg | 47 μg/kg i.v. | 0.6x |
| | | i.v.¹ | | |
| A4 | 1:2 | 19 μg/kg | 2.8 μg/kg i.v. | 7x |
| | | i.v.¹ | | |

¹tail flick, mouse

Transferred hyperalgesia

| | Ratio | | ED₅₀ visceral | Enhancement of |
|---|---|---|---|---|
| Substance | ORL1/μ | ED₅₀ acute | pain | action factor |
| B1 (fentanyl) | <1:100 | 30 μg/kg | 42 μg/kg i.v. | 0.7x |
| | | i.v.¹ | | |
| A4 | 1:2 | 19 μg/kg | 3.0 μg/kg i.v. | 6x |
| | | i.v.¹ | | |

¹tail flick, mouse

The analgesic efficacy of A4 in relation to both the tested pain parameters is higher by a factor of approx. 6 to 7 than against acute pain. In contrast, the analgesic efficacy of fentanyl against visceral inflammatory pain is lower than against acute pain.

### Action in Acute Pain Models

The mixed ORL1/μ-agonists with an ORL1:μ-ratio of 1:10 to 30:1 exhibit full efficacy in acute pain models (tail flick, mouse and rat). The results for tail flick testing are shown in Table 3 (see above). The effect is shown with reference to examples between ORL1:μ of 1:10 to 20:1. In accordance with their binding affinity for μ-OR, their effectiveness is within the range of standard opioids (sufentanil, fentanyl, buprenorphine, oxycodone, morphine) (see FIG. 13).

### Opioid-Induced Hyperalgesia

Chronic administration of opioids leads to hyperalgesia in pain patients (cf. Chu et al. 2006, J. Pain 7:43-48). A similar phenomenon also occurs after acute administration in the withdrawal situation (Angst et al. 2003, Pain 106: 49-57). In an animal model, pure μ-opioids induce transient hyperalgesia after acute administration (Opioid-induced hyperalgesia. A qualitative systematic review. Angst and Clark, Anesthesiology 2006; 104:570-87), which is, for example, detectable in the soft tail flick model as a transient “pronociceptive” phase. Corresponding findings are described in the literature. This opioid-induced hyperalgesia has been demonstrated with the assistance of a modified soft tail flick model (25% intensity of thermal radiation) for pure μ-opioids (fentanyl and morphine). In contrast, no transient hyperalgesia was observed after acute administration of mixed ORL1/μ-agonists (A4 and A10) (FIGS. 14-14c).

### Determination of Physical Dependency

Testing was carried out with two models: naloxone-induced withdrawal in mice and spontaneous withdrawal in rats. In both models, withdrawal symptoms were distinctly reduced with mixed ORL1/μ-agonists in comparison with pure μ-agonists.

Jumping Test in Mice: Test for Determining Physical Dependency (Saelens J K, Arch. Int Pharmacodyn 190: 213-218, 1971)

The test substances are administered intraperitoneally in total 7× over two days. 5 administrations took place on the first day at around 09:00, 10:00, 11:00, 13:00 and 15:00 and on the second day at around 09:00 and 11:00. The first 3 administrations are given in rising dosages (dosage scheme) and thereafter at the dosage of the third administration. 2 hours after the final substance administration, withdrawal is precipitated with naloxone 30 mg/kg (i.p.). The animals are then immediately individually placed in transparent observation boxes (height 40 cm, diameter 15 cm) and the jumping responses counted over 15 minutes for 5 minute periods in each case. Morphine is also administered in one dosage as a comparison/standard. Withdrawal is quantified by counting the number of jumps 0 to 10 min. after administration of naloxone. The number of animals per group with more than 10 jumps/10 min is determined and recorded as “% positive animals”. The average jump frequency in the group is also calculated. 12 animals are used per group.

μ-Agonists B1-B4 induce distinct withdrawal jumping. The μ-agonist B7 (L-methadone, levomethadone, FIG. 15) induces withdrawal jumping which is reduced in comparison with B1-B4 but is still significant. B8 and A1 also trigger significant withdrawal jumping in this test (FIGS. 15a and 15b). A9, in contrast, triggers only slight withdrawal jumping which is completely suppressed at higher dosages (FIG. 15c). After administration of A4 or A7, virtually no or no significant withdrawal jumping occurs (FIGS. 15d and 15e).

### Spontaneous Withdrawal in Rats:

The study into spontaneous opiate withdrawal was carried out in 5 phases.

Phase 1 (chronic treatment phase): rats are treated with the test substance over 3 weeks. Administration was made intraperitoneally 2 or 3× daily (depending on duration of action of the test substance).

Phase 2 (spontaneous withdrawal): Spontaneous withdrawal and a treatment-free period (phase 3) of one week then followed. The animals then received the test substance for one more week (phase 4).

Phase 5 (naloxone-induced withdrawal): withdrawal was then initiated with naloxone (10 mg/kg i.p.)

Measurement parameters in withdrawal: animal weights, behavioral parameters:

Assessment of the (6) main symptoms during withdrawal: tremor, salivation, writhing, wet dog shaking, hopping and jumping, tooth grinding

0=not present, 1=slight, 2=severe

maximum score=12

Morphine was also administered as reference substance

The study into spontaneous opiate withdrawal was designed in accordance with a description in the literature: Jaffe J H (1990) Drug addiction and drug abuse. In: Goodman Gilman A, Rall T W, Nies A S, Taylor P (eds.) The pharmacological basis of therapeutics, New York, Pergamon Press: 522-573. Blasig J, Herz A, Reinhold K, Zieglgänsberger S (1973) Development of physical dependence on morphine in respect to time and dosage and quantification of the precipitated withdrawal syndrome, Psychopharmacology 33: 19-38.

The spontaneous withdrawal results are shown in FIG. 16.

In the jumping test, A4, A7 and A9 exhibit no withdrawal symptoms or withdrawal symptoms which are at least distinctly reduced in comparison with morphine. A1 (ORL1:μ 1:10) brings about withdrawal behavior in the withdrawal jumping test, but no significant weight loss is observed during spontaneous withdrawal. With prior administration of morphine, however, the rats undergo an approx. 10% drop in body weight. A1 is thus distinguished by a reduced potential for dependency in comparison with morphine.

### Reduction of μ-Mediated Respiratory Depression by an ORL1-Dependent Mechanism

Acute μ-Mediated Respiratory Depression in Rats Method for pCO₂ and pO₂ Measurement in Rats (Blood Gas Analysis)

The respiratory depressive action of test substances is investigated after i.v. administration to instrumented, conscious rats. The test parameter is the change in carbon dioxide partial pressure (pCO₂) and oxygen partial pressure (pO₂) in arterial blood after substance administration.

Test animals: Male Sprague-Dawley rats; weight: 250-275 g

Test preparation: At least 6 days before administration of the test substance, a PP catheter is implanted under pentobarbital anaesthesia in the femoral artery and in the jugular vein of the rats. The catheters are filled with heparin solution (4000 I.U.) and closed with a wire rod.

Performance of test: The substance or vehicle is administered via the venous catheter. Before administration of the substance or vehicle and at defined times after administration of the substance or vehicle, the arterial catheter is opened and flushed with approx. 500 μl of heparin solution. Approx. 100 μl of blood are then taken from the catheter and drawn up by means of a heparinized glass capillary. The catheter is flushed once more with heparin solution and closed again. The arterial blood is immediately measured with the assistance of a blood gas analyzer (ABL 5, Radiometer GmbH, Willich, Germany).

After a minimum wash-out time of one week, the animals may again be included in the test.

Test evaluation: The blood gas analyzer automatically provides the pCO₂ and pO₂ values of the blood in mmHg. The effects of the substance on partial pressure are calculated as percentage changes relative to the preliminary values without substance or vehicle. For the purposes of statistical evaluation, the measurements after substance administration and the simultaneous measurements after vehicle administration are compared by means of one-factor analysis of variance. If a significant substance effect is found, a post hoc Dunnett's test is carried out.

In the case of pure μ-opioids (in this case fentanyl and oxycodone, FIGS. 17 and 17a), a distinct increase in arterial pCO₂ occurs at the time of maximum analgesic action due to the μ-induced respiratory depression. At a 90-100% effective dose, the pCO₂ value rises by more than 50%.

The pCO₂ value with mixed ORL1/μ-agonists was determined by way of comparison therewith. Even at a dosage which is maximally analgesically active over several hours, the arterial pCO₂ rises only by approx. 20-30% after administration of the mixed ORL1/μ-agonists (FIGS. 17b-17e).

The cause of the observed effect was investigated taking A4 by way of example. To this end, at time 0 B11 (2.15 mg/kg) was administered (i.v.) together with A4 in order to antagonize the ORL1 component and only leave the μ-effect to be observed. In a further experiment, 20 minutes after administration of A4+B11, naloxone (1 mg/kg i.v.) was administered in order to test whether the resultant respiratory depressive effect is exclusively a μ-mediated effect.

The result shows that the respiratory depression of A4, which is reduced in comparison with pure μ-opioids, is quite clearly attributable to the ORL1 component (FIG. 18). Accordingly, after antagonization with B11, the pCO₂ value rises to a value which is typical of pure μ-opioids. If naloxone is administered after the maximum pCO₂ increase has been reached, the value drops back down. This demonstrates that μ-mediated respiratory depression is reduced by the ORL1 component.

### Safety Margin

The safety margins for various mixed ORL1/μ-agonists and pure μ-agonists, presented as the margin between threshold dose (ED₁₀) for an increase in arterial pCO₂ and the semimaximal active dosage in the Chung model (ED₅₀) are shown in FIG. 19.

In A1, A4, A5 and A7, the threshold dose (ED₁₀) for an increase in arterial pCO₂ is higher by a factor of approximately 3 to 20 than the semimaximal active dosage (ED₅₀) in the Chung model, whereas the threshold dose for the μ-agonists B1, B3, and B5 is of the same range as the semimaximal active dosage (ED₅₀) in the Chung model, or, in the case of B4, even distinctly lower. The safety margins between action and side-effect are therefore distinctly larger for mixed ORL1/μ-agonists in comparison with μ-agonists.

### Psychological Dependency/Addiction

With regard to the investigation of place preference see: Tzschentke, T. M., Bruckmann, W. and Friderichs, F. (2002) Lack of sensitization during place conditioning in rats is consistent with the low abuse potential of tramadol, Neuroscience Letters 329, 25-28.

A4, A6 and A7 induce place preference, but, in comparison with the pure μ-antagonists B1 and B3-B5, in a dose range which is lower by a factor of up to 100 (FIG. 20).

It has been shown, taking A7 by way of example, that the reduced place preference in this case is attributable to the ORL1 component. Place preference was first of all tested at different dosages (FIG. 21).

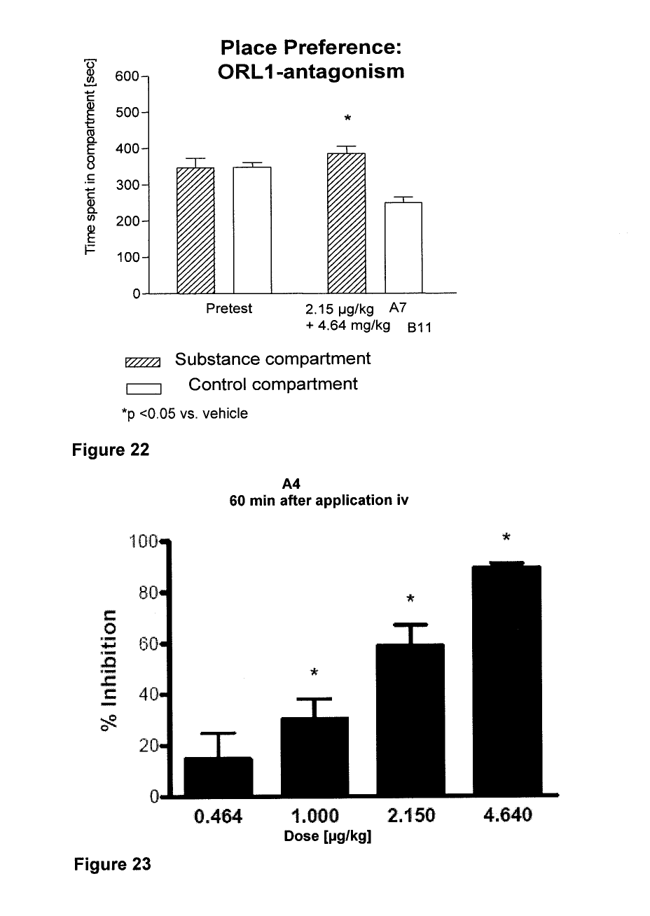

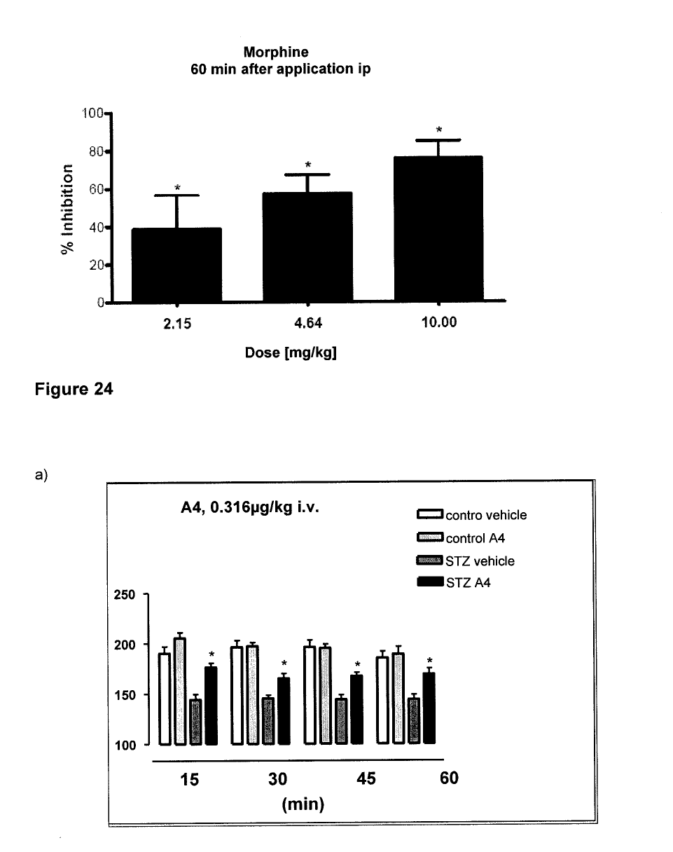

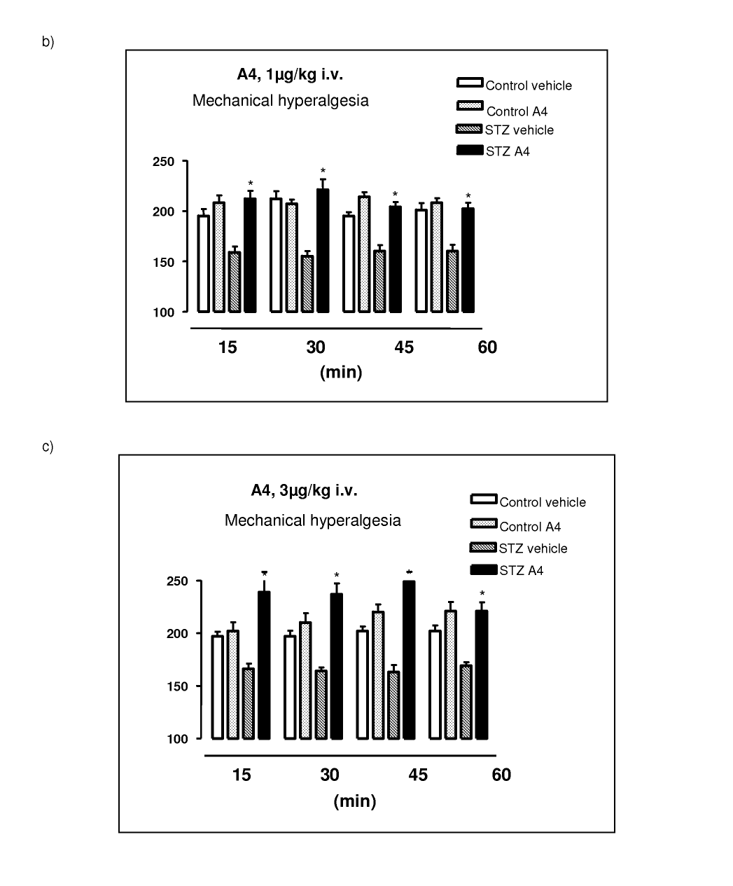

After administration of A7, antagonization was performed with B11. It proved possible to show that, once the ORL1 component had been blocked, the threshold for induction of a place preference is shifted towards lower dosages (FIG. 22). This finding demonstrates that the ORL1 component attenuates μ-OR-mediated place conditioning.

While the present invention has been described in conjunction with the specific embodiments set forth above, many alternatives, modifications and other variations thereof will be apparent to those of ordinary skill in the art. The preceding description of the invention, therefore, is not meant to limit the scope of the invention in any respect. Rather, all such alternatives, modifications and variations are intended to fall within the spirit and scope of the present invention, and the scope of the invention is to be determined only by the appended issued claims and their equivalents.

1. A method for the treatment of diabetic polyneuropathy pain in a patient in need of such treatment, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor (ORL1/μ) defined as 1/[K_{i(ORL1)}/K_{i(μ)}] is from 0.1 to 30.

2. The method according to claim 1, wherein said at least one compound is a metabolite formed in vivo after a precursor thereof is administered to said patient.

3. The method according to claim 1, wherein the ratio ORL1/μ is from 0.1 to 20.

4. A method for the treatment of pain in a patient in need of such treatment and at increased risk of developing hyperalgesia, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor (ORL1/μ) defined as 1/[K_{i(ORL1)}/K_{i(μ)}] is from 0.1 to 30.

5. The method according to claim 4, wherein the patient is one selected from the group consisting of irritable colon patients, tumor pain patients and patients with musculoskeletal pain.

6. The method according to claim 4, wherein the compound or precursor thereof is used for anaesthesia or for analgesia during anaesthesia.

7. The method according to claim 4, wherein said at least one compound is a metabolite formed in vivo after a precursor thereof is administered to said patient.

8. The method according to claim 4, wherein the ratio ORL1/μ is from 0.1 to 20.

9. A method for the treatment of pain in a patient in need of such treatment and over 60 years of age, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor ORL1/μ defined as 1/[K_{i(ORL1)}/K_{i(μ)}] is from 0.1 to 30.

10. The method according to claim 9, wherein the compound or precursor thereof is used in anaesthesia.

11. The method according to claim 9, wherein said at least one compound is a metabolite formed in vivo after a precursor thereof is administered to said patient.

12. The method according to claim 9, wherein the ratio ORL1/μ is from 0.1 to 20.

13. A method for the treatment of pain in a patient in need of such treatment and having an elevated potential for addiction, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor ORL1/μ defined as 1/[K_{i(ORL1)}/K_{i(μ)}] is from 0.1 to 30.

14. The method according to claim 13, wherein the patient suffers from a psychological disorder.

15. The method according to claim 13, wherein said at least one compound is a metabolite formed in vivo after a precursor thereof is administered to said patient.

16. The method according to claim 13, wherein the ratio ORL1/μ is from 0.1 to 20.

17. A method for the treatment of pain as a consequence of an inflammatory disease in a patient in need of such treatment, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor ORL1/μ defined as 1/[K_{i(ORL1)}/K_{i(μ)}] is from 0.1 to 30.

18. The method according to claim 17, wherein said at least one compound is a metabolite formed in vivo after a precursor thereof is administered to said patient.

19. The method according to claim 17, wherein the ratio ORL1/μ is from 0.1 to 20.

20. A method for the treatment of pain in a patient in need of such treatment, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor ORL1/μ defined as 1/[K_{i(ORL1)}/K_{i(μ)}] is from 0.1 to 30.

21. The method according to claim 20, wherein the pain is chronic pain.

22. The method according to claim 21, wherein the chronic pain is neuropathic pain.

23. The method according to claim 22, wherein the neuropathic pain is pain with postzoster neuralgia.

24. The method according to claim 22, wherein the compound or precursor thereof is administered to said patient at a dosage which is below a dosage necessary to treat said patient for acute pain.

25. The method according to claim 24, wherein the compound or precursor thereof is administered at a dosage which is lower by a factor of at least 2 than the dosage necessary to treat said patient for acute pain.

26. The method according to claim 25, wherein the compound or precursor thereof is administered at a dosage which is lower by a factor of at least 5 than the dosage necessary to treat said patient for acute pain.

27. The method according to claim 20, wherein said at least one compound is a metabolite formed in vivo after a precursor thereof is administered to said patient.

28. The method according to claim 20, wherein the ratio ORL1/μ is from 0.1 to 20.

29. A method for the treatment of postoperative pain in a patient in need of such treatment, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor ORL1/μ defined as 1/[K_{i(ORL1)}/K_{i(μ)}] is from 0.1 to 30.

30. The method according to claim 29, wherein said at least one compound is a metabolite formed in vivo after a precursor thereof is administered to said patient.

31. The method according to claim 29, wherein the ratio ORL1/μ is from 0.1 to 20.

32. A method for the treatment of pain in a patient in need of such treatment, said method comprising administering to said patient an effective amount therefor of a mixture of a) a first compound or first precursor thereof that converts to said first compound in vivo and b) a second compound or second precursor thereof that converts to said second compound in vivo, wherein said first compound is a μ-agonist which is more selective than ORL1/μ defined as 1/[K_{i(ORL1)}/K_{i(μ)}] 0.1, and said second compound is an ORL1 agonist which is more selective than ORL1/μ defined as 1/[K_{i(ORL1)}/K_{i(μ)}] 30.

33. A method for the treatment of one or more of postoperative pain or pain with postzoster neuralgia in a patient in need of such treatment, said method comprising administering to said patient an effective amount therefor of at least one compound or a precursor thereof that converts to said at least one compound in vivo, wherein said at least one compound exhibits an affinity for the μ-opioid receptor of at least 100 nM (Kᵢ value, human) and an affinity for the ORL-1 receptor, wherein the ratio between the affinity for the ORL-1 receptor and the affinity for the μ-opioid receptor ORL1/μ defined as 1/[K_{i(ORL1)}/K_{i(μ)}] is from 0.1 to 30, and said at least one compound is selected from the group consisting of spirocyclic cyclohexane derivatives of the formula I: 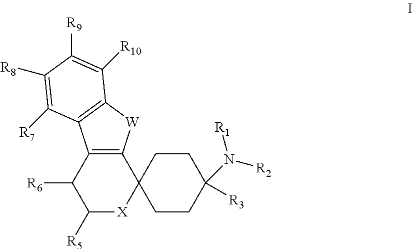
in which
R¹ and R² mutually independently denote H or CH₃, wherein R¹ and R² do not simultaneously denote H;
R³ denotes phenyl, benzyl or heteroaryl, in each case unsubstituted or monosubstituted or polysubstituted with F, Cl, OH, CN and/or OCH₃;
W denotes NR⁴, O or S;
and
R⁴ denotes H; C₁₋₅ alkyl; phenyl; phenyl-C₁₋₃-alkyl; R¹²OC—C₁₋₃-alkyl, SO₂R¹²,
wherein R¹² denotes H; C₁₋₇ aliphatic hydrocarbyl, which is branched or unbranched, saturated or unsaturated, and unsubstituted or monosubstituted or polysubstituted with OH, F and/or COOC₁₋₄ alkyl; C₄₋₆ cycloalkyl; aryl or heteroaryl, which is unsubstituted or monosubstituted or polysubstituted with F, Cl, Br, CF₃, OCH₃ and/or C₁₋₄ alkyl, which alkyl is branched or unbranched, and unsubstituted or monosubstituted or polysubstituted with F, Cl, CN, CF₃, N(CH₃)₂ and/or OH; or phenyl or heteroaryl, which is unsubstituted or monosubstituted or polysubstituted with F, Cl, Br, CF₃, OCH₃ and/or C₁₋₄ alkyl, which alkyl is branched or unbranched, wherein the phenyl or heteroaryl is attached via saturated or unsaturated C₁₋₃ aliphatic hydrocarbyl; or C₅₋₆ cycloalkyl attached via saturated or unsaturated C₁₋₃ aliphatic hydrocarbyl; OR¹³; or NR¹⁴R¹⁵;
R⁵ denotes H; COOR¹³, CONR¹³, OR¹³; C₁₋₅ aliphatic hydrocarbyl, which is saturated or unsaturated, branched or unbranched, and unsubstituted or monosubstituted or polysubstituted with OH, F, CF₃ and/or CN;
R⁶ denotes H;
or R⁵ and R⁶ together denote (CH₂)ₙ with n=2, 3, 4, 5 or 6, wherein individual hydrogen atoms may be replaced by F, Cl, NO₂, CF₃, OR¹³, CN and/or C₁₋₅ alkyl;
R⁷, R⁸, R⁹ and R¹⁰ mutually independently denote
H, F, Cl, Br, NO₂, CF₃, OH, OCH₃, CN, COOR¹³, NR¹⁴R¹⁵; or C₁₋₅ alkyl; or heteroaryl, which is unsubstituted or monosubstituted or polysubstituted with benzyl, CH₃, Cl, F, OCH₃ and/or OH;
wherein R¹³ denotes H or C₁₋₅ alkyl;
R¹⁴ and R¹⁵ mutually independently denote H or C₁₋₅ alkyl;
X denotes O, S, SO, SO₂ or NR¹⁷;
R₁₇ denotes H; C₁₋₅ aliphatic hydrocarbyl, which is saturated or unsaturated, and branched or unbranched; COR¹² or SO₂R¹²,
wherein said at least one compound or precursor thereof is optionally in the form of a pure diastereomer thereof, a racemate thereof, a pure enantiomer thereof, or in the form of a mixture of stereoisomers thereof in any desired mixing ratio;
and/or
said at least one compound or precursor thereof is in the form of a base or salt thereof.

34. The method according to claim 33, wherein said at least one compound or precursor thereof is selected from the group consisting of:
1,1-(3-methylamino-3-phenylpentamethylene)-6-fluoro-1,3,4,9-tetrahydropyrano[3,4b]indole hemicitrate;
1,1-(3-methylamino-3-phenylpentamethylene)-1,3,4,9-tetrahydropyrano[3,4-b]indole hemicitrate;
1,1-[3-dimethylamino-3-(3-thienyl)pentamethylene]-1,3,4,9-tetrahydropyrano[3,4-b]indole hemicitrate;
1,1-(3-dimethylamino-3-phenylpentamethylene)-6-fluoro-1,3,4,9-tetrahydropyrano[3,4-b]indole hemicitrate;
1,1-[3-methylamino-3-(2-thienyl)pentamethylene]-1,3,4,9-tetrahydropyrano[3,4-b]-6-fluoroindole citrate;
1,1-[3-dimethylamino-3-(2-thienyl)pentamethylene]-1,3,4,9-tetrahydropyrano[3,4-b]-6-fluoroindole hemicitrate;
1,1-[3-dimethylamino-3-(2-thienyl)pentamethylene]-1,3,4,9-tetrahydropyrano[3,4b]indole citrate; 1,1-[3-dimethylamino-3-(3-thienyl)pentamethylene]-1,3,4,9-tetrahydropyrano[3,4-b]-6-fluoroindole hemicitrate;
1,1-(3-dimethylamino-3-phenylpentamethylene)-1,3,4,9-tetrahydropyrano[3,4-b]indole hemicitrate; and
1,1-[3-methylamino-3-(2-thienyl)pentamethylene]-1,3,4,9-tetrahydropyrano[3,4-b]indole citrate.

35. (canceled)

36. The method according to claim 34, which is for the treatment of postoperative pain.

37. The method according to claim 34, which is for the treatment of pain with postzoster neuralgia.

38. A method for the treatment of pain in a patient in need of such treatment and at a heightened risk for respiratory depression, said method comprising administering to said patient an effective amount therefor of at least one compound or a precursor thereof that converts to said at least one compound in vivo, wherein said at least one compound exhibits an affinity of at least 100 nM for the μ-opioid receptor and for the ORL1 receptor and, due to the ORL1 component, induces respiratory depression which is reduced in comparison with a μ-opioid having the same affinity for the μ-opioid receptor.

39. The method according to claim 38, wherein said at least one compound is a metabolite formed in vivo after a precursor thereof is administered to said patient.

40. The method according to claim 38, wherein the at least one compound or precursor thereof exhibits a ratio ORL1/μ of from 0.1 to 20.

41. A method for the treatment of palliative pain in a patient in need of such treatment, said method comprising administering to said patient an effective amount therefor of at least one compound or a precursor thereof that converts to said at least one compound in vivo, wherein said at least one compound exhibits an affinity for the t-opioid receptor of at least 100 nM (Kᵢ value, human) and an affinity for the ORL-1 receptor, wherein the ratio between the affinity for the ORL-1 receptor and the affinity for the μ-opioid receptor (ORL1/μ) defined as 1/[K_{i(ORL1)}/K_{i(μ)}] is from 0.1 to 30.

42. The method according to claim 41, wherein said at least one compound is a metabolite formed in vivo after a precursor thereof is administered to said patient.

43. The method according to claim 41, wherein the ratio ORL1/μ is from 0.1 to 20.
